(19) United States
(12) Patent Application Publication
(10) Pub. No.: US 2014/0342381 A1
(43) Pub. Date: Nov. 20, 2014

(54) DEVICES AND METHODS FOR BIOMARKER DETECTION PROCESS AND ASSAY OF NEUROLOGICAL CONDITION

(71) Applicant: BANYAN BIOMARKERS, INC., Alachua, FL (US)

(72) Inventor: Ronald L. Hayes, Alachua, FL (US)

(73) Assignee: BANYAN BIOMARKERS, INC., Alachua, FL (US)

(21) Appl. No.: 14/217,119

(22) Filed: Mar. 17, 2014

Related U.S. Application Data
(63) Continuation-in-part of application No. 13/058,748, filed on Feb. 11, 2011.
(60) Provisional application No. 61/798,146, filed on Mar. 15, 2013.
(60) Provisional application No. 61/218,727, filed on Jun. 19, 2009.
(60) Provisional application No. 61/097,622, filed on Sep. 17, 2008.
(60) Provisional application No. 61/188,554, filed on Aug. 11, 2008.

(51) Int. Cl. G01N 33/543

(52) U.S. Cl. CPC G01N 33/54386
USPC 435/7.94; 435/287.2; 435/7.92

(57) ABSTRACT

The present invention relates to an exemplary in vitro diagnostic (IVD) device used to detect the presence of and/or severity of neural injuries or neuronal disorders in a subject. The IVD device relies on an immunoassay which identifies biomarkers that are diagnostic of neural injury and/or neuronal disorders in a biological sample, such as whole blood, plasma, serum, cerebrospinal fluid (CSF). The inventive IVD device may measure one or more of several neural specific markers in a biological sample and output the results to a machine readable format wither to a display device or to a storage device internal or external to the IVD.

### CROSS-REFERENCE TO RELATED APPLICATIONS

The present application is a non-provisional application that claims priority of U.S. Provisional patent application Ser. No. 61/798,146 filed on Mar. 15, 2013; and is a continuation-in-part of U.S. non-provisional application Ser. No. 13/058,748 filed Feb. 11, 2011; that in turn is a US national phase application of PCT/US09/53376 filed Aug. 11, 2009; that in turn claims priority benefit to Provisional application No. 61/218,727, filed on Jun. 19, 2009, provisional application No. 61/097,622, filed on Sep. 17, 2008, provisional application No. 61/188,554, filed on Aug. 11, 2008; the content of which is herein incorporated by reference.

### GOVERNMENT SUPPORT

This invention was made with government support under W81XWH-10-C-0251 awarded by the Department of Defense and USA MED RESEARCH ACQ/ACTIVITY. The government has certain rights in the invention.

### FIELD OF THE INVENTION

The invention provides for an in vitro diagnostic device and software which enables the reliable detection of damage to the nervous system (central nervous system (CNS) and peripheral nervous system (PNS)), brain injury and neural disorders of an individual through biomarker identification. These devices and software methods provide simple yet sensitive clinical approaches to diagnosing damage to the central nervous system, neurotoxicity, brain injury and neuronal disorders using biological fluid particularly measuring for one or more of a biomarker such as glial fibrillary acidic protein (GFAP) or one of its breakdown products, Ubiquitin carboxyl-terminal hydrolase L1 (UCH-L1), neuron specific enolase (NSE), Microtubule-associated protein 2 (MAP2), myelin basic protein (MBP), α-II spectrin breakdown product (SBDP) preferably SBDP150, SBDP150i SBDP145, SBDP120, S100 calcium binding protein B (S100b), vesicular membrane protein neurensin-1 (p24), collapsin response mediated proteins (CRMP's) and breakdown products thereof, or synaptotagmin and breakdown products thereof. Inventive markers include proteins; or protein fragments; auto-antibodies; DNA; RNA; or miRNA.

### BACKGROUND OF THE INVENTION

The field of clinical neurology remains frustrated by the recognition that secondary injury to a central nervous system tissue associated with physiologic response to the initial insult could be lessened if only the initial insult could be rapidly diagnosed or in the case of a progressive disorder before stress on central nervous system tissues reached a preselected threshold. Traumatic, ischemic, and neurotoxic chemical insult, along with generic disorders, all present the prospect of brain damage. Traumatic, ischemic, and neurotoxic chemical insult also present the prospect of brain or other neurological damage. While the diagnosis of severe forms of each of these causes is straightforward through clinical response testing, computed tomography (CT), and magnetic resonance imaging (MRI), the imaging diagnostics are limited by both the high cost of spectroscopic imaging and long diagnostic time. The clinical response testing of incapacitated individuals is of limited value and often precludes a nuanced diagnosis. Additionally, owing to the limitations of existing diagnostics, situations arise wherein a subject experiences a stress to their neurological condition but are often unaware that damage has occurred or fail seek treatment as the subtle symptoms often quickly resolve. The lack of treatment of these mild to moderate challenges to neurologic condition of a subject can have a cumulative effect or otherwise result in a severe brain damage event, either of which have a poor clinical prognosis.

In order to overcome the limitations associated with spectroscopic and clinical response diagnosis of neurological condition, there is increasing attention on the use of biomarkers as internal indicators of change to molecular or cellular level health condition of a subject. As biomarker detection uses a sample obtained from a subject, typically cerebrospinal fluid, blood, or plasma, and detects the biomarkers in that sample, biomarker detection holds the prospect of inexpensive, rapid, and objective measurement of neurological condition. The attainment of rapid and objective indicators of neurological condition allows one to determine severity of a non-normal brain condition with a previously unrealized degree of objectivity, predict outcome, and guide therapy of the condition, as well as monitor subject responsiveness and recovery. Additionally, such information as obtained from numerous subjects allows one to gain a degree of insight into the mechanism of brain injury.

A number of biomarkers have been identified as being associated with severe traumatic brain injury as is often seen in vehicle collision and combat wounded subjects. These biomarkers included spectrin breakdown products such as SBDP150, SBDP150i, SBDP145 (calpain mediated acute neural necrosis), SBDP120 (caspase mediated delayed neural apoptosis), UCH-L1 (neuronal cell body damage marker), and MAP2 dendritic cell injury associated marker. The nature of these biomarkers is detailed in U.S. Pat. Nos. 7,291,710 and 7,396,654, the contents of which are hereby incorporated by reference. Other biomarkers may be used to detect for neural injury, neuronal disease, or neural disorder, T disclosures presented in US 2007/0003982 A1, US 2005/0260697 A1, US 2009/0317805 A1, US 2005/0260654 A1, US 2009/0087868 A1, US 2010/0317041 A1, US 2011/0177974 A1, US 2010/0047817 A1, US 2012/0196307 A1, US 2011/0082203 A1, US 2011/0097392 A1, US 2011/0143375 A1, US 2013/0029859 A1, US 2012/0202231 A1, and US 2013/0022982 A1, the contents of which are also hereby incorporated by reference.

Several biomarkers of neurotoxicity have also been presented; see US 2013/0029362 A1, the contents of which are also hereby incorporated by reference. Biomarkers of neurotoxicity include ubiquitin carboxyl-terminal hydrolase-L1 (UCH-L1); spectrin; a spectrin breakdown product (SBDP); MAP1, MAP2; GFAP, ubiquitin carboxyl-terminal esterase; ubiquitin carboxyl-terminal hydrolase; a neuronally-localized intracellular protein; MAP-tau; C-tau; Poly (ADP-ribose) polymerase (PARD); a collapsin response mediator protein, synaptotagmin, βIII-tubulin, S100β; neuron-specific enolase, neurofilament protein light chain, nestin, α-internexin; breakdown products thereof, post-translationally modified forms thereof, derivatives thereof, and combinations thereof.

Thus, there exists a need for a process and an assay for providing improved measurement of neurological condition, neurotoxicity, or cell damage through the quantification of biomarkers in the clinical environment, whether alone or in combination with another biomarker associated with the specific condition.

There is also an unmet need for clinical intervention through the use of an in vitro diagnostic device to identify these neurochemical markers so that subject results may be obtained rapidly in any medical setting to direct the proper course of treatment for subjects suffering from a neural injury or neuronal disorder.

### SUMMARY OF THE INVENTION

The present invention provides an in vitro diagnostic device specifically designed and calibrated to detect protein markers that are differentially present in the samples of patients suffering from neural injury and/or neuronal disorders, neurotoxicity, or nerve cell damage. These devices present a sensitive, quick, and non-invasive method to aid in diagnosis of neural injury and/or neuronal disorders by detecting and determining the amount of biomarkers that are indicative to the respective injury type. The measurement of these markers, alone or in combination of other markers for the injury type, in patient samples provides information that a diagnostician can correlate with a probable diagnosis of the extent of an injury such as traumatic brain injury (TBI) and/or stroke.

In certain inventive embodiments, the invention provides an in vitro diagnostic device to measure biomarkers that are indicative of traumatic brain injury, stroke, Alzheimer's disease, epilepsy, hypoxic ischemic encephalopathy (HIE), chronic traumatic encephalopathy (CTE), neural disorders, brain damage, neural damage due to drug or alcohol addiction, or other diseases and disorders associated with the brain or nervous system, such as the central nervous system or peripheral nervous system. In certain inventive embodiments, the biomarkers are proteins, fragments or derivatives thereof, and are associated with neuronal cells, brain cells or any cell that is present in the brain, central nervous system, and peripheral nervous system.

In other inventive embodiments, neural proteins, peptides, fragments or derivatives thereof which are detected by an assay. An inventive in vitro diagnostic device further includes a process for determining the neurological condition of a subject or cells from the subject includes measuring a sample obtained from the subject or cells from the subject at a first time for a quantity of a first biomarker selected from the group of GFAP, UCH-L1, NSE, S100B, MBP, MAP2, SBDP, CRMP, synaptotagmin, or neurensin-1 (p24). The sample is also measured for a quantity of at least one additional neuroactive biomarker. Through comparison of the quantity of the first biomarker and the quantity of the at least one additional neuroactive biomarker to normal levels for each biomarker, the neurological condition of the subject and its severity is determined. A ratio is readily calculated of the concentration of two or more biomarkers collected from a sample at a given time. The ratio is then compared with concentration of the two or more biomarkers at a later time to provide clinically relevant information such as the type of neural tissues injured, severity of injury, the effectiveness of a therapy, or a combination thereof. It is appreciated that the biomarker data of the present invention is readily supplemented with conventional data such as intra-cranial pressure, CT scan data, MRI scan data, and combinations thereof.

An inventive in vitro diagnostic device necessarily incorporates an assay for determining the neurological condition of a subject or neural cells from the subject is also provided. The assay includes at least a first biomarker specifically binding agent wherein a first biomarker is one of GFAP, UCH-L1, NSE, S100B, MBP, MAP2, SBDP, CRMP, synaptotagmin, or neurensin-1 (p24). In certain inventive embodiments, an assay is incorporated which may detect one or more markers selected from the group of GFAP, UCH-L1, NSE, S100B, MBP, MAP2, SBDP, CRMP, synaptotagmin, or neurensin-1 (p24).

The inventive device also provides a process for determining if a subject has suffered mild traumatic brain injury or moderate traumatic brain injury in an event is provided that includes measuring a sample obtained from the subject or cells from the subject at a first time after the event for a quantity of GFAP or by measuring for GFAP and at least one other biomarker selected from UCH-L1, NSE, S100B, MBP, MAP2, SBDP, CRMP, synaptotagmin, or neurensin-1 (p24). GFAP and UCH-L1 have a particular synergy for diagnosing mild and moderate TBI, thus by comparing the quantity of GFAP and UCH-L1 to each other and to a metric of what level is expected in a non-injured subject, using an algorithm of the assay output and a pre-programmed comparison metric, which has been clinically validated, a device interpolates the data to determine if the subject has suffered an injury, determine the severity of injury (mild, moderate, severe) and by including even more markers, may determine the time after injury and predict a recovery outcome. A comparison of these markers may also be used to determine other neurological disorders such as Alzheimer's, Parkinson's disease, and may predict other neural injuries using this or any number of additional biomarkers, such as neurotoxicity such as is disclosed in WO/2011/123844 and whose disclosure is incorporated herein by reference.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 represents UCHL1 levels in serum following TBI at various timepoints;

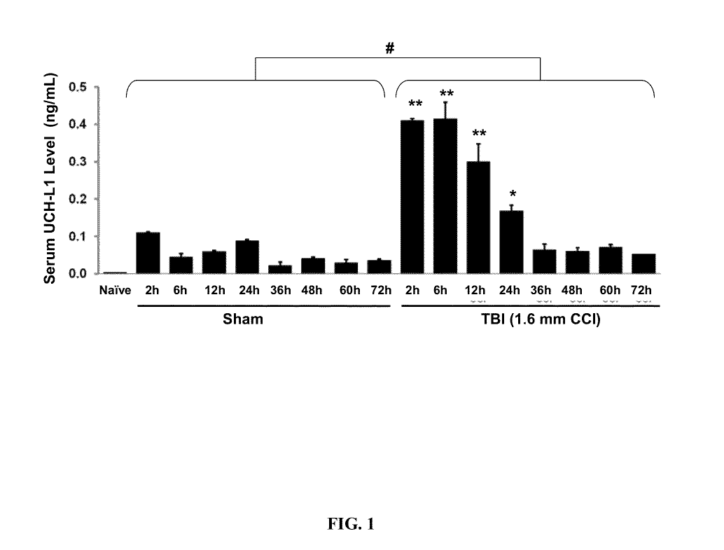

FIG. 2 represents the effect of dicyclomine on SBDPs in CSF following CCI;

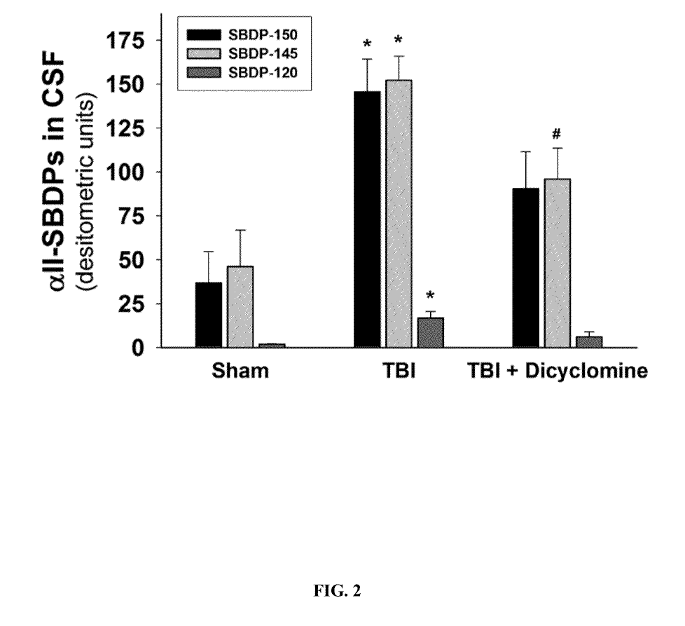

FIG. 3 represents MAP2 elevation in CSF and serum following sham, mild MCAO challenge, and severe MCAO challenge

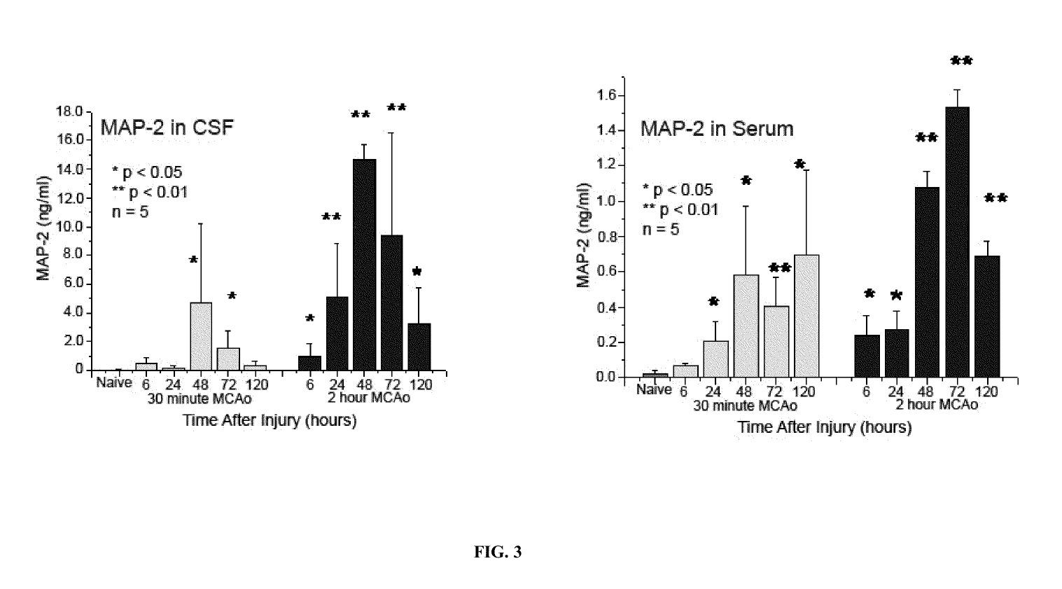

FIG. 4 are bar graphs of GFAP and other biomarkers for human control and severe TBI subjects from CSF samples;

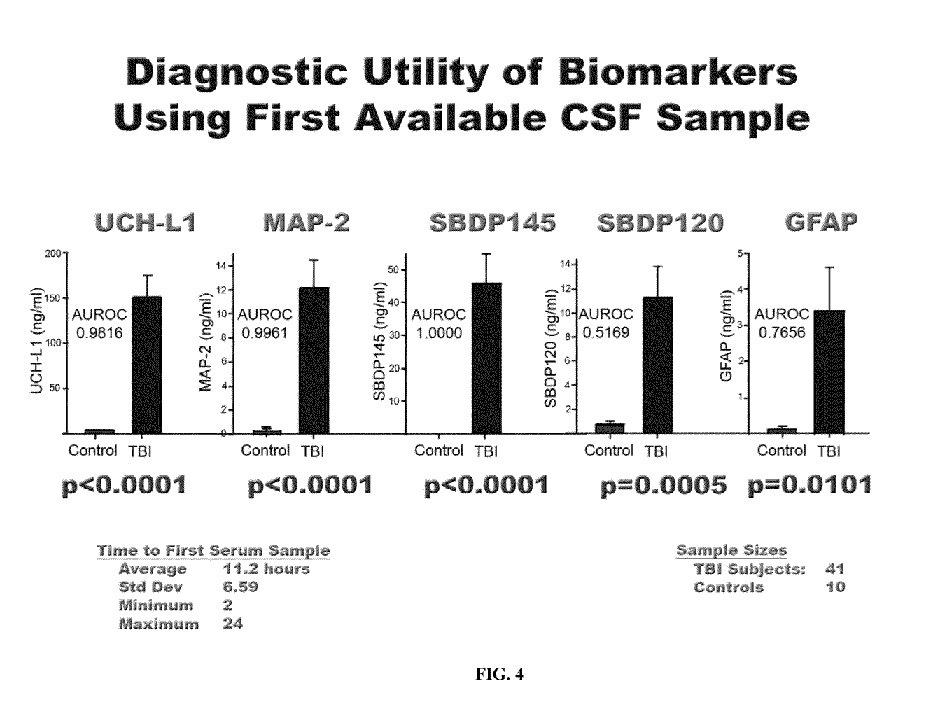

FIG. 5 are bar graphs of GFAP and other biomarkers for human control and severe TBI subjects of FIG. 1 from serum samples;

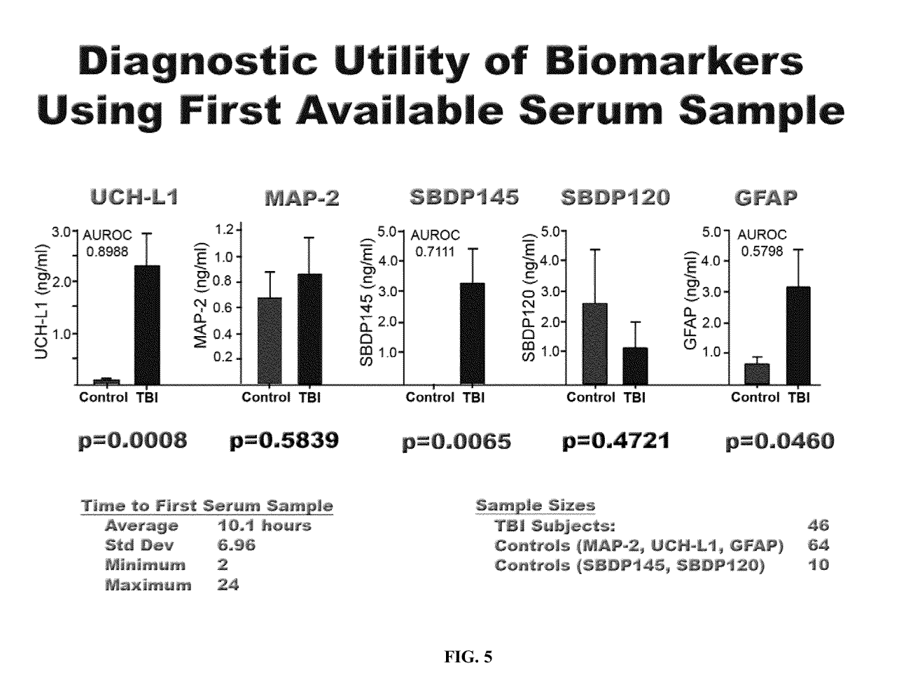

FIG. 6 are bar graphs of GFAP and other biomarkers for human control and severe TBI subjects summarizing the data of FIGS. 4 and 5;

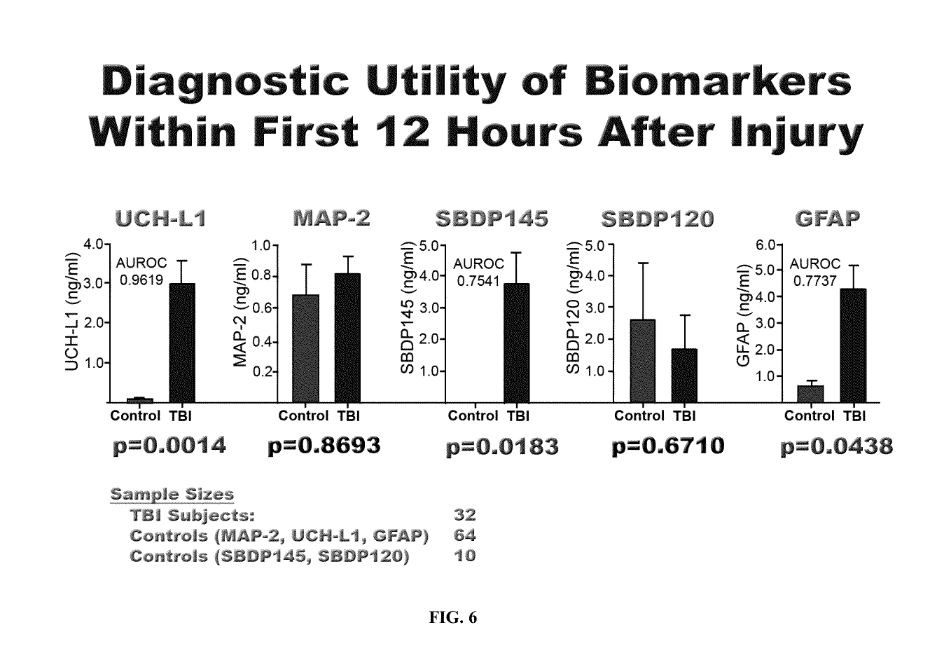

FIG. 7 are plots of inventive biomarkers from CSF and serum samples from another individual human subject of traumatic brain injury as a function of time;

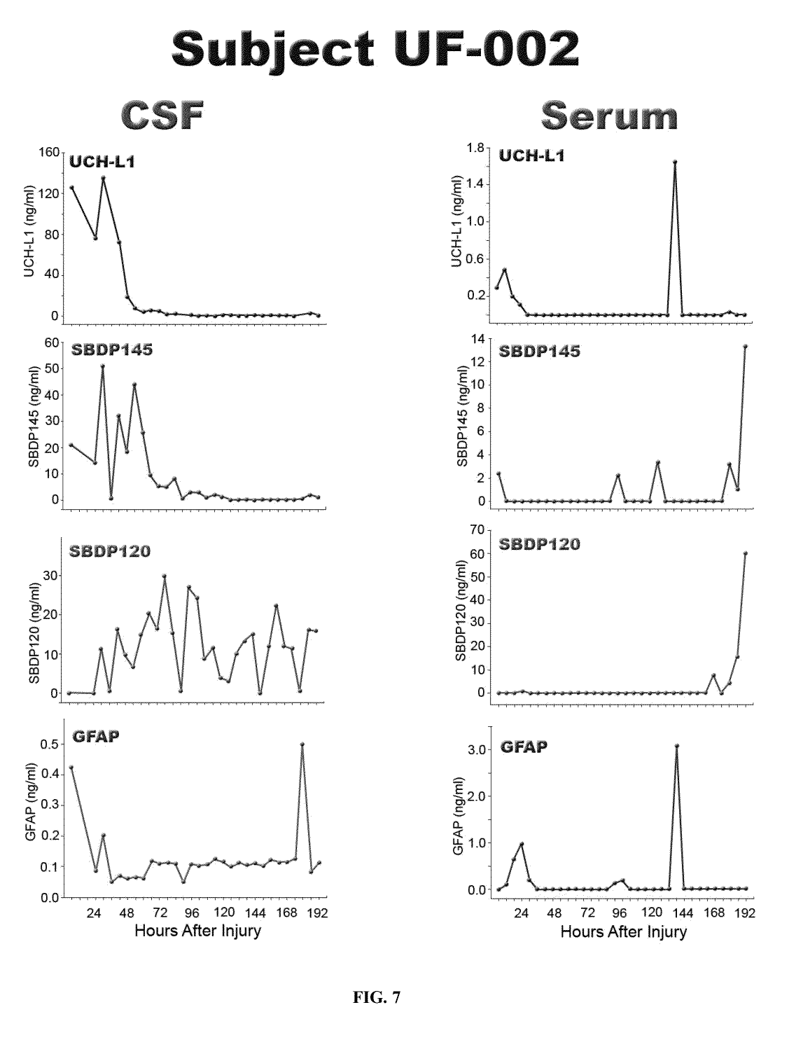

FIG. 8 are bar graphs of GFAP concentration for controls, as well as individuals in the mild/moderate traumatic brain injury cohort as a function of CT scan results upon admission and 24 hours thereafter;

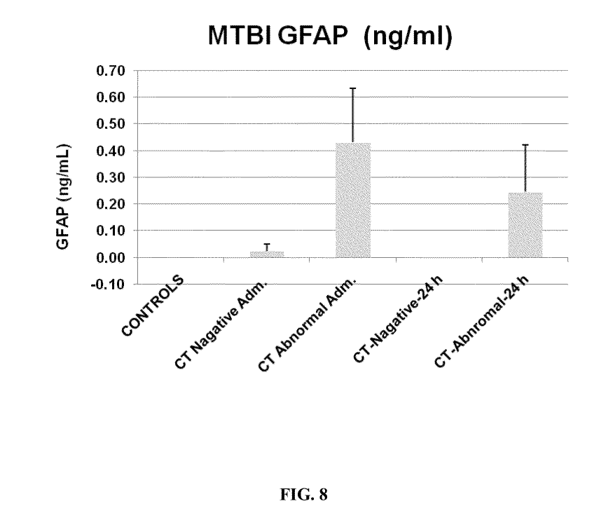

FIG. 9 are bar graphs of parallel assays for UCH-L1 biomarker from the samples used for FIG. 8;

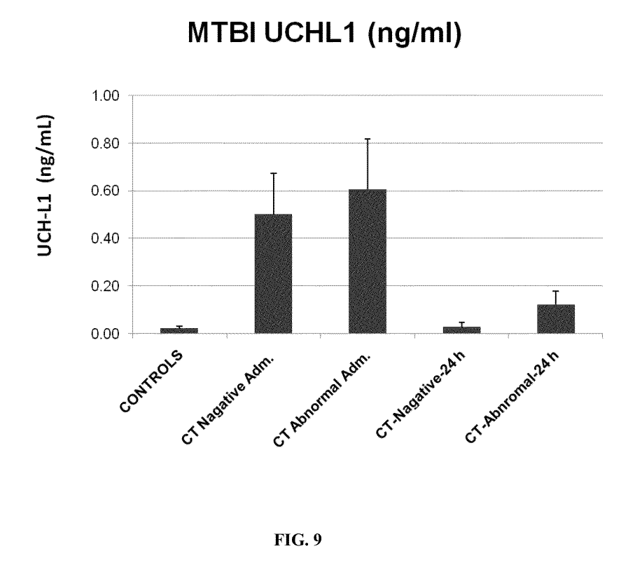

FIG. 10 are bar graphs showing the concentration of UCH-L1 and GFAP as well as a biomarker not selected for diagnosis of neurological condition, S100 beta, provided as a function of injury magnitude between control, mild, and moderate traumatic brain injury;

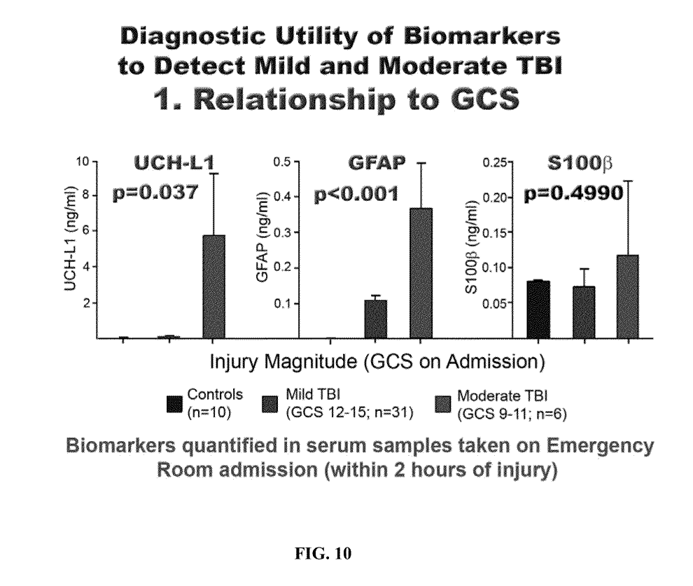

FIG. 11 are bar graphs showing the concentration of the same markers as depicted in FIG. 10 with respect to initial evidence upon hospital admission as to lesions in tomography scans;

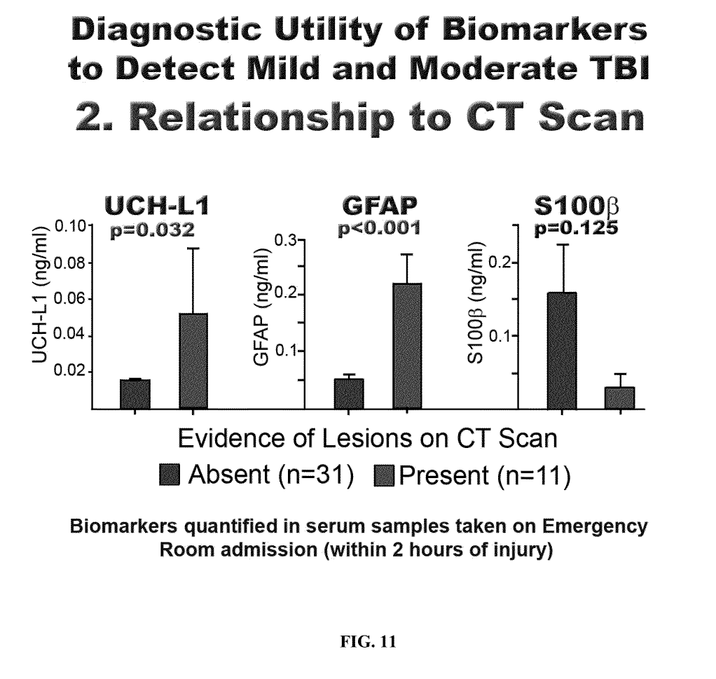

FIG. 12 represents biomarker levels in human subjects with varying types of brain injury;

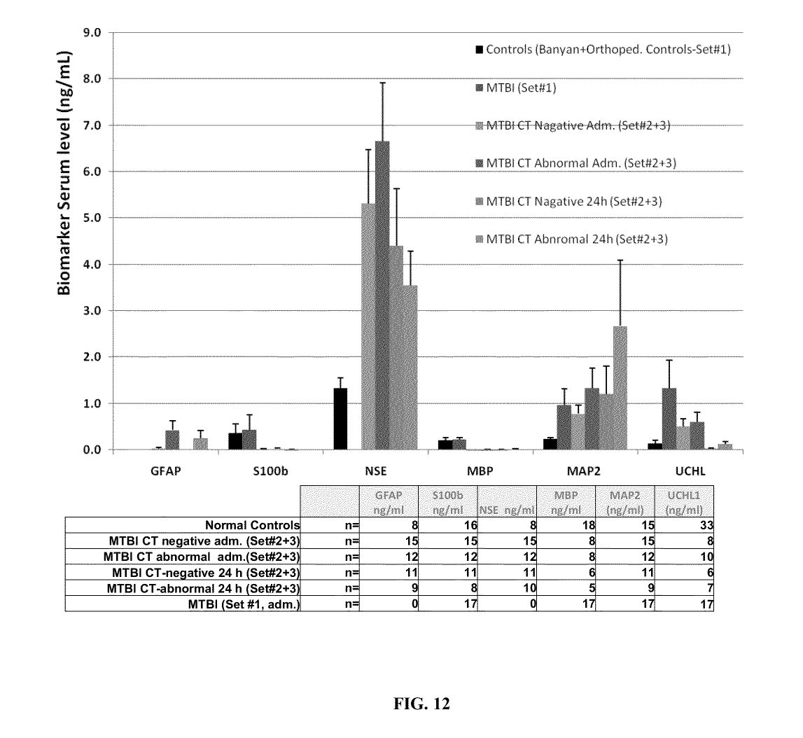

FIG. 13 are plots that represent ROC analysis of UCH-L1, GFAP and SBDP145 in human CSF (severe TBI vs. Control A) First 24 hours post-injury;

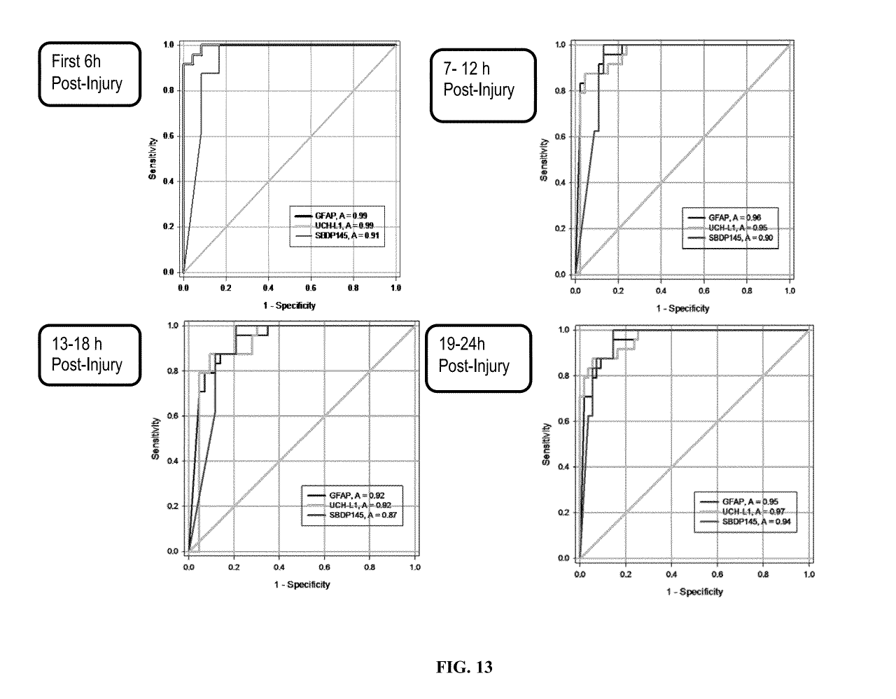

FIG. 14 is a plot that represent ROC analysis of UCH-L1 and GFAP in human CSF (mild TBI vs. normal Controls) a mean of 3h35′ with a range 15′-14h35 post-injury.

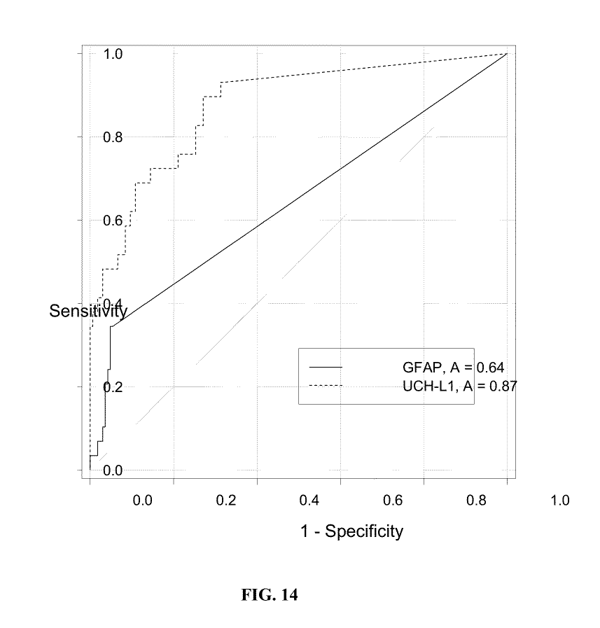

FIG. 15 are bar graphs of showing the elevation of brain injury biomarkers (GFAP, UCH-L1 and MAP2) in plasma from stroke patients.

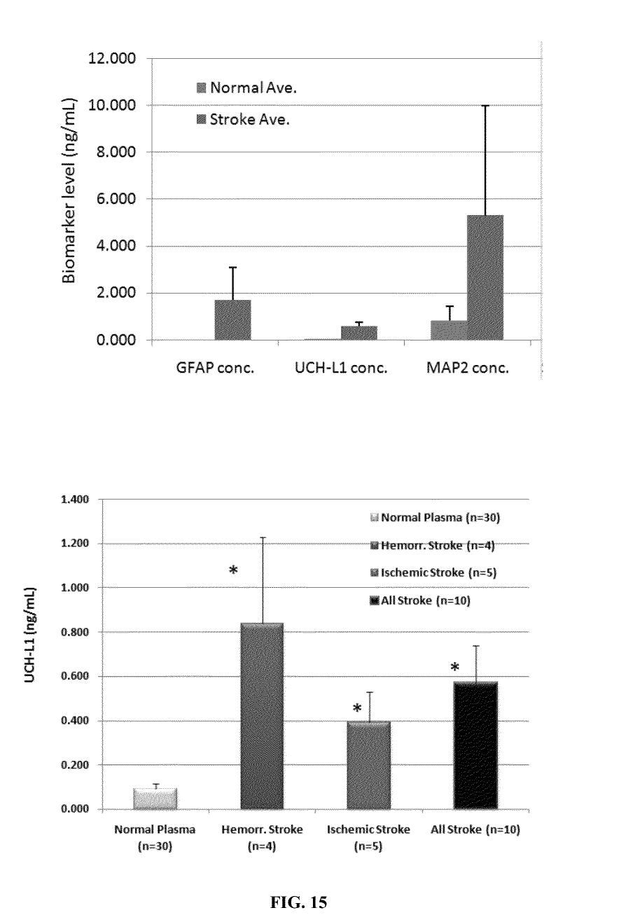

FIG. 16 illustrates rat cerebrocortical cultures challenged by various agents and probed for Tau and TBDPs;

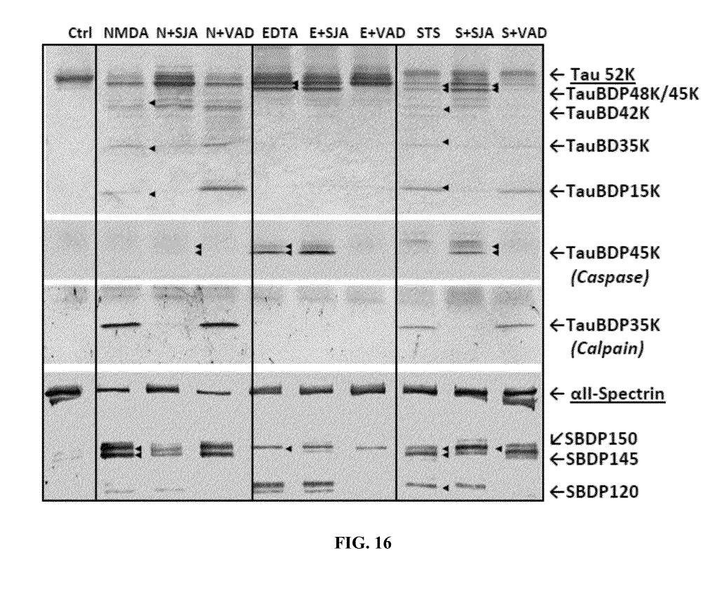

FIG. 17 are western blots of human CSF for GFAP and GBDPs in two patients (A and B) at various time points following injury;

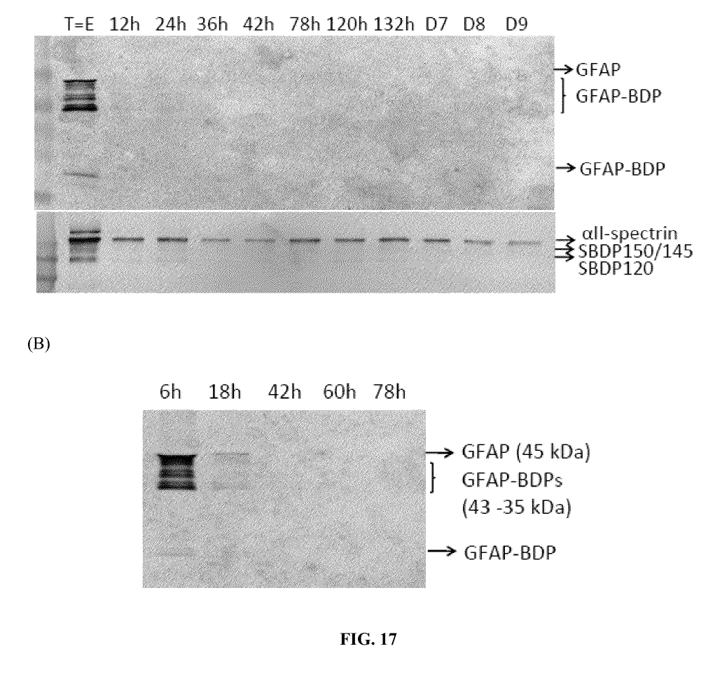

FIG. 18 illustrates the presence of human serum autoantibodies directed to brain specific proteins found in post-mortem human brain protein lysate where (A) is total protein load measured by Coomassie brilliant blue stain, (B) western blot probing with control serum, or (C) western blot probing with pooled post-TBI patient serum;

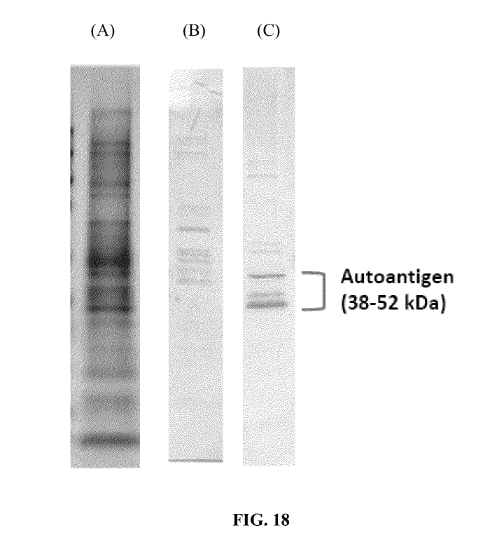

FIG. 19 illustrates the presence of autoantibodies in serum from human subjects at 72 hours and 30 days post-TBI;

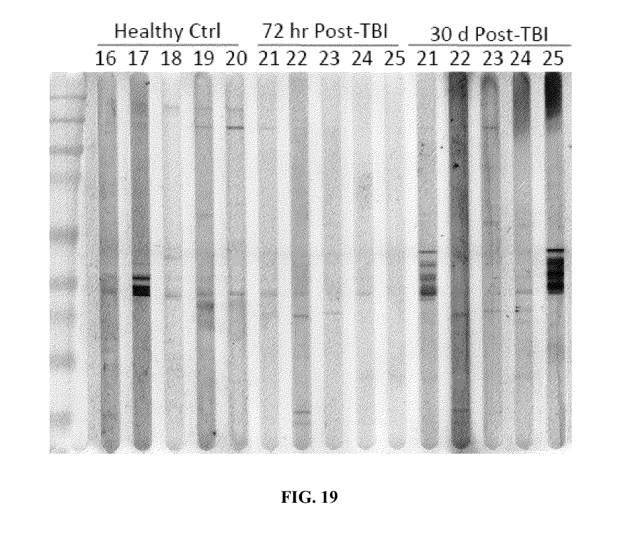

FIG. 20 illustrates the presence of autoantibody in the serum from a human subject detectable within 5 days following TBI (A) and their IgG specificity is confirmed (B);

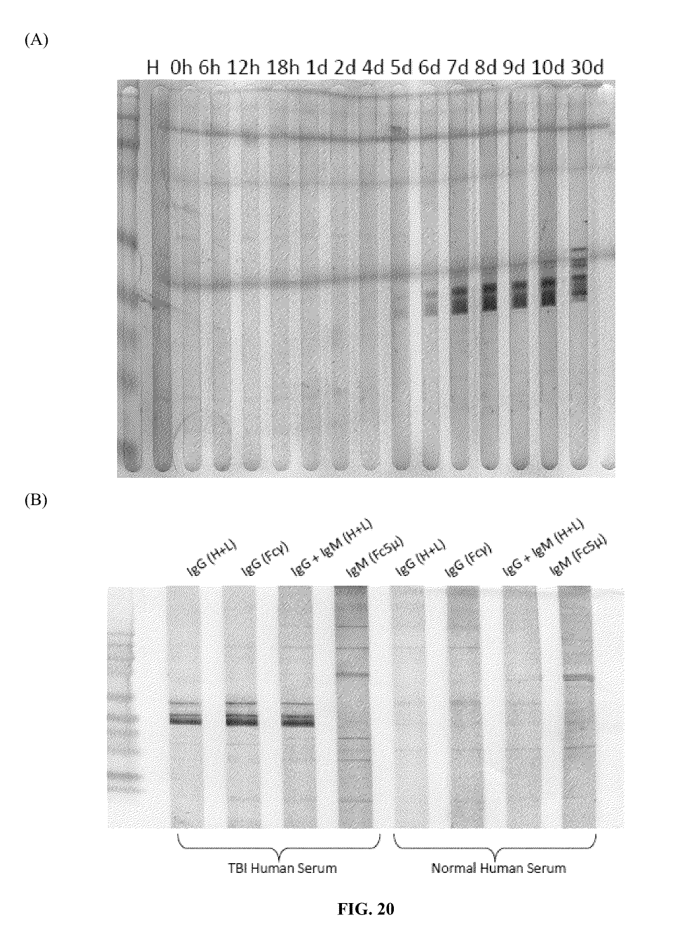

FIG. 21 illustrates that the TBI induced autoantigens are brain specific;

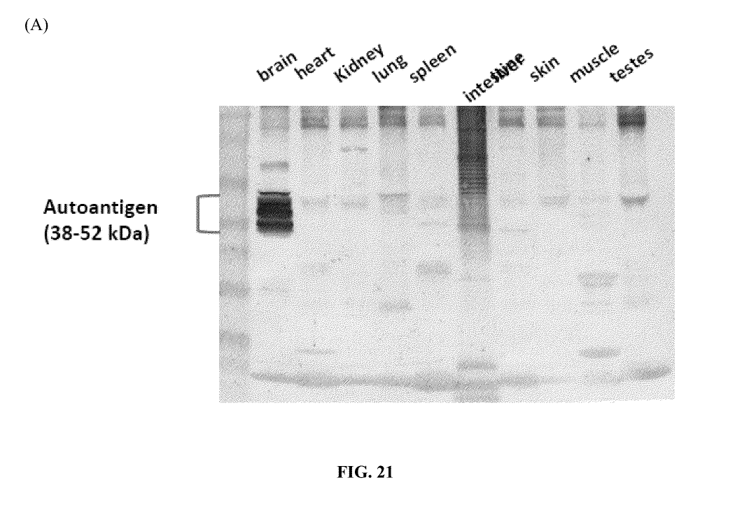

FIG. 22 illustrates the presence of autoantibodies to GFAP, neurofascin, and MBP in serum from a human TBI subject obtained 10 days following injury;

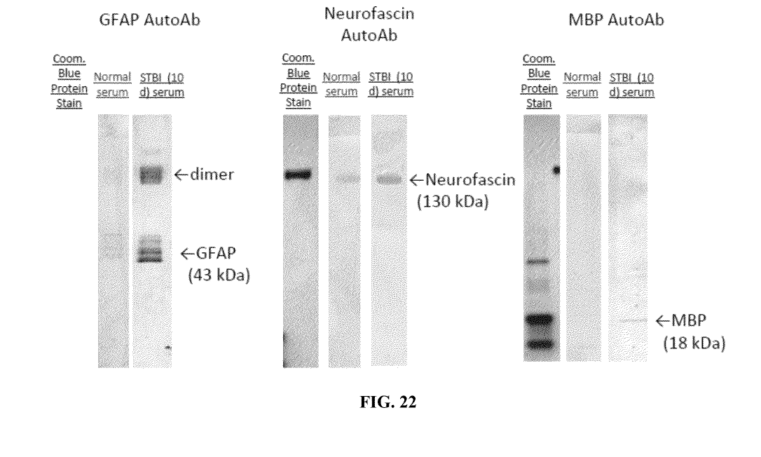

FIG. 23 illustrates the level of GFAP, UCH-L1 and S100β in serum from TBI human subjects with mild and moderate injury magnitude;

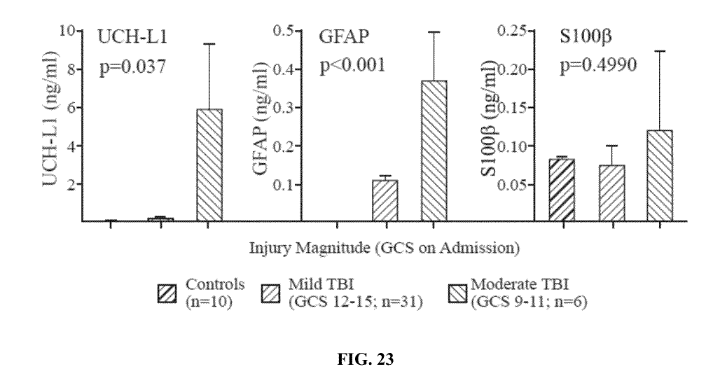

FIG. 24 is a schematic view of the in vitro diagnostic device.

FIG. 25 represents UCH-L1, GFAP, S100β, NSE, MBP, and MAP2 amounts present in serum post severe traumatic brain injury in human subjects as a function of CT scan results;

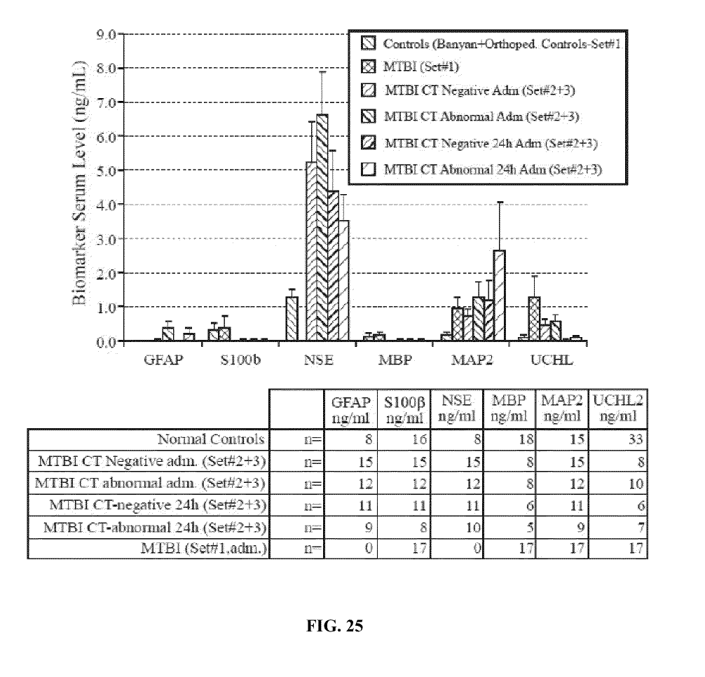

FIG. 26 illustrates relative CNPase expression in rat cortex (A) and hippocampus (B) following experimental blast-induced non-penetrating injury;

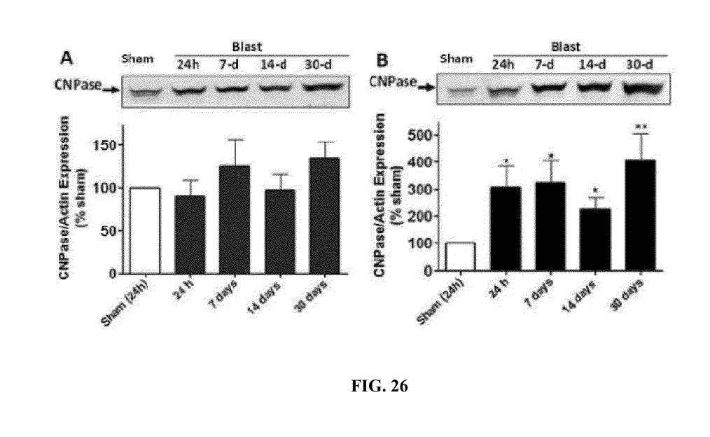

FIG. 27 illustrates NSE levels in rat CSF (A) and serum (B) as measured by ELISA following experimental blast-induced non-penetrating injury;

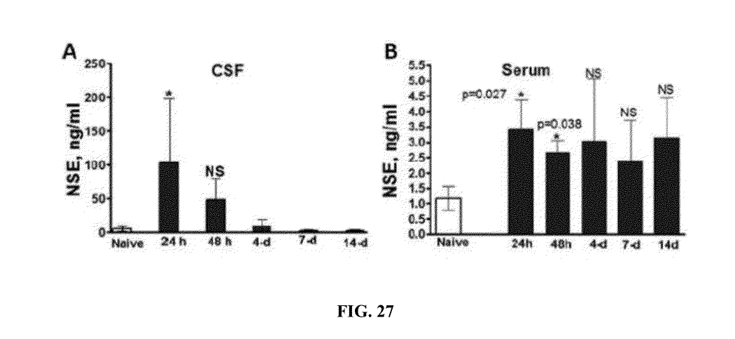

FIG. 28 illustrates Neurotoxicity biomarker elevation in biofluid compartment following neurotoxic response to Methamphetamine or cisplatin.

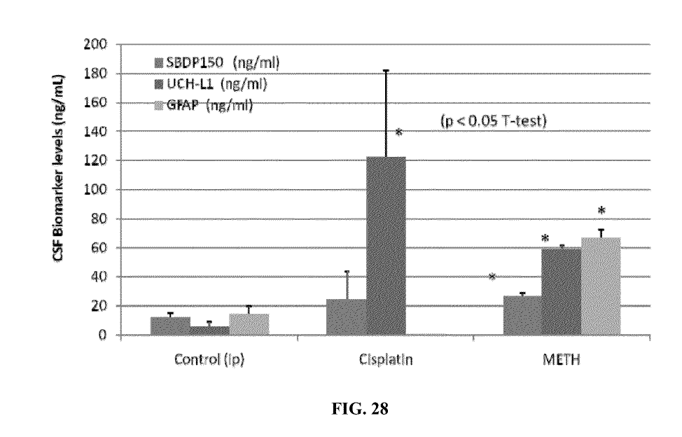

FIG. 29 depicts the time dependent effect of KA (9 mg/Kg) administration on spectrin breakdown products in the rat CSF.

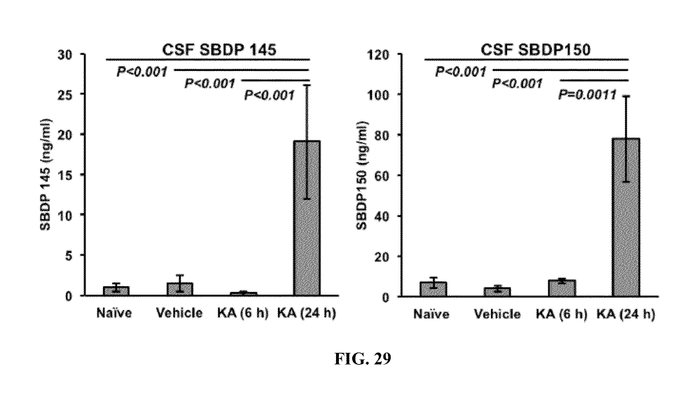

### DETAILED DESCRIPTION OF THE PREFERRED EMBODIMENTS

The present invention has utility in the diagnosis and management of abnormal neurological condition. Through the measurement of biomarkers such as GFAP, UCH-L1, NSE, S100B, MBP, MAP2, SBDP, CRMP, CNPase, NRP-2 synaptotagmin, or neurensin-1 (p24) from a subject alone or in combination, a determination of subject neurological condition is provided with greater specificity than previously attainable. The present description is directed toward a first biomarker of GFAP for illustrative purposes only and is not meant to be a limitation on the practice or scope of the present invention. It is appreciated that the invention encompasses several other first and additional biomarkers illustratively including UCH-L1, NSE, MAP2, and SBDP. The description is appreciated by one of ordinary skill in the art as fully encompassing all inventive biomarkers as an inventive first biomarker as described herein. Surprisingly, by combining the detection of more than one biomarker, a synergistic result is achieved. Illustratively, combining the detection of two neuroactive biomarkers such as UCH-L1 and GFAP provides sensitive detection that is unexpectedly able to discern the level and severity of an abnormal neurological condition in a subject.

The present invention further incorporates by reference the disclosures presented in US 2007/0003982 A1, US 2005/0260697 A1, US 2009/0317805 A1, US 2005/0260654 A1, US 2009/0087868 A1, US 2010/0317041 A1, US 2011/0177974 A1, US 2010/0047817 A1, US 2012/0196307 A1, US 2011/0082203 A1, US 2011/0097392 A1, US 2011/0143375 A1, US 2013/0029859 A1, US 2012/0202231 A1, and US 2013/0022982 A1. The in vitro diagnostic devices described herein have incorporated assays contained therein, which assays may be substituted herein using the methods therein contained.

The present invention provides for the detection of a neurological condition in a subject. A neurological condition may be an abnormal neurological condition such as that caused by genetic disorder, injury, or disease to nervous tissue. As such, it is a further object of the present invention to provide a means for detecting or diagnosing an abnormal neurological condition in a subject.

The present invention also provides an assay for detecting or diagnosing the neurological condition of a subject. As the neurological condition may be the result of stress such as that from exposure to environmental, therapeutic, or investigative compounds, it is a further aspect of the present invention to provide a process and assay for screening candidate drug or other compounds or for detecting the effects of environmental contaminants regardless of whether the subject itself or cells derived there from are exposed to the drug candidate or other possible stressors.

For purposes of the subject invention, brain injury is divided into two levels, mild traumatic brain injury (MTBI), and traumatic brain injury (TBI). An intermediate level of moderate TBI is also referred to herein. The spectrum between MTBI and extending through moderate TBI is also referred to synonymously mild to moderate TBI or by the abbreviation MMTBI. TBI is defined as an injury that correlates with a two-fold increase or greater of two-fold decrease or greater in molecular marker levels or activities. MTBI is defined and an injury that correlates with less than a two-fold increase or two-fold decrease in molecular marker levels or activities.

### In Vitro Diagnostic Device

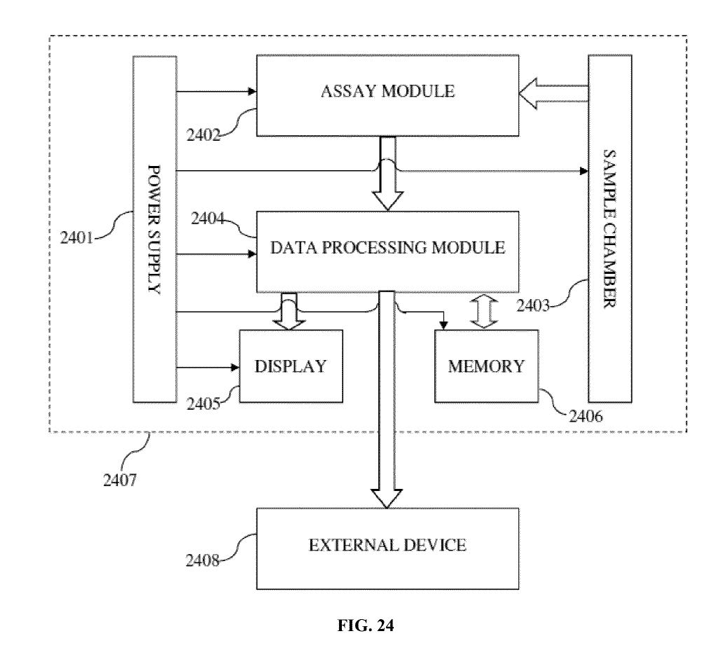

FIG. 24 schematically illustrates an inventive in vitro diagnostic device. An inventive in vitro diagnostic device comprised of at least a sample collection chamber 2403, an assay module 2402 used to detect biomarkers of neural injury or neuronal disorder, and a user interface that relates the amount of the measured biomarker measured in the assay module. The in vitro diagnostic device may comprise of a handheld device, a bench top device, or a point of care device.

The sample chamber 2403 can be of any sample collection apparatus known in the art for holding a biological fluid. In one embodiment, the sample collection chamber can accommodate any one of the biological fluids herein contemplated, such as whole blood, plasma, serum, urine, sweat or saliva.

The assay module 2402 is preferably comprised of an assay which may be used for detecting a protein antigen in a biological sample, for instance, through the use of antibodies in an immunoassay. The assay module 2402 may be comprised of any assay currently known in the art; however the assay should be optimized for the detection of neural biomarkers used for detecting neural injury or neuronal disorder in a subject. The assay module 2402 is in fluid communication with the sample collection chamber 2403. In one embodiment, the assay module 2402 is comprised of an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. In one embodiment a colorimetric assay may be used which may comprise only of a sample collection chamber 2403 and an assay module 2402 of the assay. Although not specifically shown these components are preferably housed in one assembly 2407. In one embodiment the assay module 2402 contains an agent specific for detecting one or more of the biomarkers of glial fibrillary acidic protein (GFAP) or one of its breakdown products, Ubiquitin carboxyl-terminal hydrolase L1 (UCH-L1), neuron specific enolase (NSE), Microtubule-associated protein 2 (MAP2), myelin basic protein (MBP), α-II spectrin breakdown product (SBDP) preferably SBDP150, SBDP150i SBDP145, SBDP120, S100 calcium binding protein B (S100b), collapsin response mediated proteins (CRMP's) and breakdown products thereof, or synaptotagmin and breakdown products thereof. The assay module 2402 may contain additional agents to detect additional biomarkers, as is described herein.

In another preferred embodiment, the inventive in vitro diagnostic device contains a power supply 2401, an assay module 2402, a sample chamber 2403, and a data processing module 2405. The power supply 2401 is electrically connected to the assay module and the data processing module. The assay module 2402 and the data processing module 2405 are in electrical communication with each other. As described above, the assay module 2402 may be comprised of any assay currently known in the art; however the assay should be optimized for the detection of neural biomarkers used for detecting neural injury or neuronal disorder in a subject. The assay module 2402 is in fluid communication with the sample collection chamber 2403. The assay module 2402 is comprised of an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. A biological sample is placed in the sample chamber 2403 and assayed by the assay module 2402 detecting for a biomarker of neural injury or neuronal disorder. The measured amount of the biomarker by the assay module 2402 is then electrically communicated to the data processing module 2404. The data processing 2404 module may comprise of any known data processing element known in the art, and may comprise of a chip, a central processing unit (CPU), or a software package which processes the information supplied from the assay module 2402.

In one embodiment, the data processing module 2404 is in electrical communication with a display 2405, a memory device 2406, or an external device 2408 or software package (such as laboratory and information management software (LIMS)). In one embodiment, the data processing module 2404 is used to process the data into a user defined usable format. This format comprises of the measured amount of neural biomarkers detected in the sample, indication that a neural injury or neuronal disorder is present, or indication of the severity of the neural injury or neuronal disorder. The information from the data processing module 2404 may be illustrated on the display 2405, saved in machine readable format to a memory device, or electrically communicated to an external device 2408 for additional processing or display. Although not specifically shown these components are preferably housed in one assembly 2407. In one embodiment, the data processing module 2404 may be programmed to compare the detected amount of the biomarker transmitted from the assay module 2402, to a comparator algorithm. The comparator algorithm may compare the measure amount to the user defined threshold which may be any limit useful by the user. In one embodiment, the user defined threshold is set to the amount of the biomarker measured in control subject, or a statistically significant average of a control population.

In one embodiment, the methods and in vitro diagnostic tests and products described herein may be used for the diagnosis of autism and ASD in at-risk patients, patients with non-specific symptoms possibly associated with autism, and/or patients presenting with related disorders. In another embodiment, the methods and in vitro diagnostic tests described herein may be used for screening for risk of progressing from at-risk, non-specific symptoms possibly associated with ASD, and/or fully-diagnosed ASD. In certain embodiments, the methods and in vitro diagnostic tests described herein can be used to rule out screening of diseases and disorders that share symptoms with ASD. In yet another embodiment, the methods and in vitro diagnostic tests described herein may indicate diagnostic information to be included in the current diagnostic evaluation in patients suspected of having neural injury or neuronal disorder.

In one embodiment, an in vitro diagnostic test may comprise one or more devices, tools, and equipment configured to hold or collect a biological sample from an individual. In one embodiment of an in vitro diagnostic test, tools to collect a biological sample may include one or more of a swab, a scalpel, a syringe, a scraper, a container, and other devices and reagents designed to facilitate the collection, storage, and transport of a biological sample. In one embodiment, an in vitro diagnostic test may include reagents or solutions for collecting, stabilizing, storing, and processing a biological sample. Such reagents and solutions for nucleotide collecting, stabilizing, storing, and processing are well known by those of skill in the art and may be indicated by specific methods used by an in vitro diagnostic test as described herein. In another embodiment, an in vitro diagnostic test as disclosed herein, may comprise a micro array apparatus and reagents, a flow cell apparatus and reagents, a multiplex nucleotide sequencer and reagents, and additional hardware and software necessary to assay a genetic sample for certain genetic markers and to detect and visualize certain biological markers.

### Data Processing Module

FIG. 24 further illustrates a data processing module 2404 contained within the in vitro diagnostic device. The data processing module 2404 includes of a central processing unit (CPU), a memory unit, an input/output component, and a network component. The data processing module 2404 receives instructions from software to process data received from the assay module 2402 into a user defined usable format. The information generated from the data processing module 2404 may be illustrated on the display 2405 of a graphical user interface (GUI), saved in machine readable format to the memory unit, or electrically communicated to an external device 2408 for additional processing or display by wireless or wired communication. The CPU carries out the software's instructions and dictates the data processing module's remaining components to process any inputs and signals received from the assay module 2402.

The input component receives a signal, an input, from the assay model 2402 stating the measured amount of a specific biomarker present in an analyzed sample. The data processing module 2404 receives and compares the input to a preprogrammed threshold level, predetermined for each biomarker respectively. The result of the comparison is a determination of the presence and severity of the neurological condition. The results may be saved to the memory unit for later access by the user. After the comparison, the data processing module 2404 generates an output signal of the processed measure amount based on the comparison of the input to a preprogrammed threshold.

The memory unit stores the data containing a preprogrammed threshold level for each respective biomarker. The data processing module 2404 accesses the preprogrammed threshold level to compare an input to a biomarker's proper levels. The preprogrammed threshold level is a predetermined amount based on a known positive level. In an alternative embodiment, the preprogrammed threshold may be a predetermined amount based on a known negative level. In an alternative embodiment, the preprogrammed threshold level may be an amount of the biomarker measured in normal control. The specific level for each of the embodiments is determined through prior experimentation. The memory unit also stores the results of the data processing module's comparison.

The output component relays to the display 2405 the processed measured amount, resulting from the comparison of the input to the preprogrammed threshold, in a user defined usable format. The display 2405 provides an output from which the user may determine the measured amount of the respective biomarker present in the sample, an indication of the presence or absence of neurological condition, and/or an indication of the severity of the neurological condition.

The network component electronically communicates the processed data through a wired connection to an external device at a remote location. The user may directly connect the in vitro diagnostic device to another computer to download the data stored to be saved in a separate location for additional processing or display. In an alternative embodiment, the network component may consist of a wireless feature. The wireless feature allows the user to transfer the processed data to an external device at a remote location without the need of a direct connection.

In an alternative embodiment, the data processing module 2404 may compare the levels of two or more biomarkers to determine the type of neurological condition present. The input component receives multiple inputs from a multiplex assay module. The data processing module 2404 then compares each signal to the respective biomarker's threshold level. The output component relays the processed measured amounts, resulting from the comparison, to the display 2405. The display 2405 provides an output from which the user may determine the measured amount of the respective biomarker present in the sample, an indication of the presence or absence of a neurological condition, and/or an indication of the severity of neurological condition. Additionally, through the comparison of measure amounts of multiple biomarkers, the data processing module 2404 may generate data from which the user may determine the specific type of neurological condition. The output component may also relay this data to the display 2405.

### Neural Biomarkers

The inventive in vitro diagnostic device provides the ability to detect and monitor levels of these proteins after neurotoxicity or CNS injury to provides enhanced diagnostic capability by allowing clinicians (1) to determine the level of injury severity in patients with various CNS injuries, (2) to monitor patients for signs of secondary CNS injuries that may elicit these cellular changes and (3) to continually monitor the effects of therapy by examination of these proteins in biological fluids, such as blood, plasma, serum, CSF, urine, saliva or sweat. Unlike other organ-based diseases where rapid diagnostics for surrogate biomarkers prove invaluable to the course of action taken to treat the disease, no such rapid, definitive diagnostic tests exist for traumatic or ischemic brain injury that might provide physicians with quantifiable neurochemical markers to help determine the seriousness of the injury, the anatomical and cellular pathology of the injury, and the implementation of appropriate medical management and treatment.

In certain inventive embodiments, the biological samples is one of CSF, blood, serum, plasma, sweat, saliva and urine. It should be appreciated that after injury to the nervous system (such as brain injury), the neural cell membrane is compromised, leading to the efflux of neural proteins first into the extracellular fluid or space and to the cerebrospinal fluid. Eventually the neural proteins efflux to the circulating blood (as assisted by the compromised blood brain barrier) and, through normal bodily function (such as impurity removal from the kidneys), the neural proteins migrate to other biological fluids such as urine, sweat, and saliva. Thus, other suitable biological samples include, but not limited to such cells or fluid secreted from these cells. It should also be appreciated that obtaining biological fluids such as cerebrospinal fluid, blood, plasma, serum, saliva and urine, from a subject is typically much less invasive and traumatizing than obtaining a solid tissue biopsy sample. Thus, samples, which are biological fluids, are preferred for use in the invention.

An inventive device or process, in certain inventive embodiments, includes determining the neurological condition of a subject by assaying a sample derived from a subject at a first time for the presence of a first biomarker. A biomarker is a cell, protein, nucleic acid, steroid, fatty acid, metabolite, or other differentiator useful for measurement of biological activity or response. Biomarkers operable herein illustratively include: GFAP) or one of its breakdown products, Ubiquitin carboxyl-terminal hydrolase L1 (UCH-L1), neuron specific enolase (NSE), Microtubule-associated protein 2 (MAP2), myelin basic protein (MBP), α-II spectrin breakdown product (SBDP) preferably SBDP150, SBDP150i SBDP145, SBDP120, S100 calcium binding protein B (S100b), vesicular membrane protein neurensin-1 (p24), neurofilament proteins, NF-L, NF-M, NF-H, collapsin response mediated proteins (CRMP's) and breakdown products thereof, or synaptotagmin and breakdown products thereof. Other potential biomarkers illustratively include those identified by Kobeissy, F H, et al, Molecular & Cellular Proteomics, 2006; 5:1887-1898, the contents of which are incorporated herein by reference, or others known in the art.

A first biomarker is, in certain inventive embodiments, a neuroactive biomarker. Illustrative examples of neuroactive biomarkers include GFAP, ubiquitin carboxyl-terminal esterase L1 (UCH-L1), Neuron specific enolase (NSE), spectrin breakdown products (SBDP), preferably SBDP150, SBDP150i SBDP145, SBDP120, S100 calcium binding protein B (S100b), microtubule associated proteins (MAP), preferably MAP2, MAP1, MAP3, MAP4, MAP5, myelin basic protein (MBP), Tau, Neurofilament protein (NF), Cannabinoid Receptor (CB), CAM proteins, Synaptic protein, collapsin response mediator proteins (CRMP), inducible nitric oxide synthase (iNOS), Neuronal Nuclei protein (NeuN), 2′,3′-cyclic nucleotide-3′-phosphohydrolase (CNPase), Neuroserpin, alpha-internexin, microtubule-associated protein 1 light chain 3 (LC3), Neurofascin, the glutamate transporters (EAAT), Nestin, Cortin-1,2′, and BIII-Tubulin.

The inventive process also includes assaying the sample for at least one additional neuroactive biomarker. The one additional neuroactive biomarker is in some embodiments not the same biomarker as the first biomarker and varies as to primary amino acid structure or isoform relative to the first neuroactive biomarker. Any of the aforementioned inventive biomarkers are operable as an additional neuroactive biomarker. Any number of biomarkers can be detected such as 2, 3, 4, 5, 6, 7, 8, 9, 10, or more. Detection can be either simultaneous or sequential and may be from the same biological sample or from multiple samples from the same or different subjects. In certain inventive embodiments, detection of multiple biomarkers is in the same assay chamber. The inventive process further includes comparing the quantity of the first biomarker and the quantity of the at least one additional neuroactive biomarker to normal levels of each of the first biomarker and the one additional neuroactive biomarker to determine the neurological condition of the subject.

In certain inventive embodiments, a biomarker is GFAP. GFAP is associated with glial cells such as astrocytes. Preferably, an additional neuroactive biomarker is associated with the health of a different type of cell associated with neural function. For example, CNPase is found in the myelin of the central nervous system, and NSE is found primarily in neurons. More preferably, the other cell type is an axon, neuron, or dendrite.

In another preferred embodiment, especially for MBTI and MMTBI, is UCH-L1 in combination with other biomarkers such as GFAP and MAP2.

It is appreciated however, that multiple biomarkers may be predictors of different modes or types of damage to the same cell type. Through the use of an inventive assay inclusive of biomarkers associated with glial cells as well as at least one other type of neural cell, the type of neural cells being stressed or killed as well as quantification of neurological condition results provides rapid and robust diagnosis of traumatic brain injury type. Measuring GFAP along with at least one additional neuroactive biomarker and comparing the quantity of GFAP and the additional biomarker to normal levels of the markers provides a determination of subject neurological condition.

In certain inventive embodiments, specific biomarker levels that when measured in concert with GFAP afford superior evaluation of subject neurological condition include SBDP 150, SBDP150i, a combination of SBDP145 (calpain mediated acute neural necrosis) and SBDP120 (caspase mediated delayed neural apoptosis), UCH-L1 (neuronal cell body damage marker), and MAP2. This is noted to be of particular value in measuring MMTBI and screening drug candidates or other neural cell stressor compounds with cell cultures.

In certain inventive embodiments, examples of biological samples are illustratively cells, tissues, cerebral spinal fluid (CSF), artificial CSF, whole blood, serum, plasma, cytosolic fluid, urine, feces, stomach fluids, digestive fluids, saliva, nasal or other airway fluid, vaginal fluids, semen, buffered saline, saline, water, or other biological fluid recognized in the art. Most preferably, a biological sample is CSF or blood serum. It is appreciated that two or more separate biological samples are optionally assayed to elucidate the neurological condition of the subject. It has been found that biomarkers are transmitted from CSF and blood serum to biological fluids at a predictable kinetic rate.

In addition to increased cell expression, biomarkers also appear in biological fluids in communication with injured cells. Obtaining biological fluids such as cerebrospinal fluid (CSF), blood, plasma, serum, saliva and urine, from a subject is typically much less invasive and traumatizing than obtaining a solid tissue biopsy sample. Thus, samples that are biological fluids are preferred for use in the invention. CSF, in particular, is preferred for detecting nerve damage in a subject as it is in immediate contact with the nervous system and is readily obtainable. Serum is a preferred biological sample as it is easily obtainable and presents much less risk of further injury or side-effect to a donating subject.

To provide correlations between neurological condition and measured quantities of GFAP and other neuroactive biomarkers, samples of CSF or serum are collected from subjects with the samples being subjected to measurement of GFAP as well as other neuroactive biomarkers. The subjects vary in neurological condition. Detected levels of GFAP and other neuroactive biomarkers are optionally then correlated with CT scan results as well as GCS scoring. Based on these results, an inventive assay is developed and validated (Lee et al., Pharmacological Research 23:312-328, 2006). It is appreciated that GFAP and other neuroactive biomarkers, in addition to being obtained from CSF and serum, are also readily obtained from blood, plasma, saliva, urine, as well as solid tissue biopsy. While CSF is a preferred sampling fluid owing to direct contact with the nervous system, it is appreciated that other biological fluids have advantages in being sampled for other purposes and therefore allow for inventive determination of neurological condition as part of a battery of tests performed on a single sample such as blood, plasma, serum, saliva or urine.

### Neurotoxicity Markers

In certain inventive embodiments, detection the inventive in vitro diagnostic device provides the ability to detect and monitor levels of proteins detecting a neurotoxic insult. Several biomarkers are used, each of which an assay is developed and incorporated into the inventive in vitro diagnostic devices. Biomarkers of neurotoxicity include ubiquitin carboxyl-terminal hydrolase-L1 (UCH-L1); spectrin; a spectrin breakdown product (SBDP); MAP1, MAP2; GFAP, ubiquitin carboxyl-terminal esterase; ubiquitin carboxyl-terminal hydrolase; a neuronally-localized intracellular protein; MAP-tau; C-tau; Poly (ADP-ribose) polymerase (PARP); a collapsin response mediator protein, synaptotagmin, βIII-tubulin, S100β; neuron-specific enolase, neurofilament protein light chain, nestin, α-internexin; breakdown products thereof, post-translationally modified forms thereof, derivatives thereof, and combinations thereof

### Biological Samples

Biological samples of CSF, blood, urine and saliva are collected using normal collection techniques. For example, and not to limit the sample collection to the procedures containted herein, CSF Lumbar Puncture (LP) a 20-gauge introducer needle is inserted and an amount of CSF is withdrawn. For blood, the samples may be collected by venipuncture in Vacutainer tubes, and if preferred spun down and separated into serum and plasma. For Urine and saliva, samples are collected avoiding the introduction of contaminants into the specimen is preferred. All biological samples may be stored in aliquots at −80° C. for later assay. Surgical techniques for obtaining solid tissue samples are well known in the art. For example, methods for obtaining a nervous system tissue sample are described in standard neuro-surgery texts such as Atlas of Neurosurgery: Basic Approaches to Cranial and Vascular Procedures, by F. Meyer, Churchill Livingstone, 1999; Stereotactic and Image Directed Surgery of Brain Tumors, 1st ed., by David G. T. Thomas, WB Saunders Co., 1993; and Cranial Microsurgery: Approaches and Techniques, by L. N. Sekhar and E. De Oliveira, 1st ed., Thieme Medical Publishing, 1999. Methods for obtaining and analyzing brain tissue are also described in Belay et al., Arch. Neurol. 58: 1673-1678 (2001); and Seijo et al., J. Clin. Microbiol. 38: 3892-3895 (2000). Any suitable biological samples can be obtained from a subject to detect markers. It should be appreciated that the methods employed herein may be identically reproduced for any biological fluid to detect a marker or markers in a sample.

After insult, the damaged tissue, organs, or nerve cells in in vitro culture or in situ in a subject express altered levels or activities of one or more proteins than do such cells not subjected to the insult. Thus, samples that contain nerve cells, e.g., a biopsy of a central nervous system or peripheral nervous system tissue are illustratively suitable biological samples for use in the invention. In addition to nerve cells, however, other cells express illustratively αII-spectrin including, for example, cardiomyocytes, myocytes in skeletal muscles, hepatocytes, kidney cells and cells in testis. A biological sample including such cells or fluid secreted from these cells might also be used in an adaptation of the inventive methods to determine and/or characterize an injury to such non-nerve cells.

A subject illustratively includes a dog, a cat, a horse, a cow, a pig, a sheep, a goat, a chicken, non-human primate, a human, a rat, and a mouse. Subjects who most benefit from the present invention are those suspected of having or at risk for developing abnormal neurological conditions, such as victims of brain injury caused by traumatic insults (e.g., gunshot wounds, automobile accidents, sports accidents, shaken baby syndrome), ischemic events (e.g., stroke, cerebral hemorrhage, cardiac arrest), neurodegenerative disorders (such as Alzheimer's, Huntington's, and Parkinson's diseases; prion-related disease; other forms of dementia), epilepsy, substance abuse (e.g., from amphetamines, Ecstasy/MDMA, or ethanol), and peripheral nervous system pathologies such as diabetic neuropathy, chemotherapy-induced neuropathy and neuropathic pain.

Baseline levels of several biomarkers are those levels obtained in the target biological sample in the species of desired subject in the absence of a known neurological condition. These levels need not be expressed in hard concentrations, but may instead be known from parallel control experiments and expressed in terms of fluorescent units, density units, and the like. Typically, baselines are determined from subjects where there is an absence of a biomarker or present in biological samples at a negligible amount. However, some proteins may be expressed less in an injured patient. Determining the baseline levels of protein biomarkers in a particular species is well within the skill of the art. Similarly, determining the concentration of baseline levels of neural injury biomarkers is well within the skill of the art.

### Immunoassays

The inventive in vitro diagnostic device makes use of an assay module 402, which may be one of many types of assays. The biomarkers of the invention can be detected in a sample by any means. Methods for detecting the biomarkers are described in detail in the materials and methods and Examples which follow. For example, immunoassays, include but are not limited to competitive and non-competitive assay systems using techniques such as western blots, radioimmunoassays, ELISA (enzyme linked immunosorbent assay), “sandwich” immunoassays, magnetic immunoassays, radioisotope immunoassay, fluorescent immunoassays, immunoprecipitation assays, precipitin reactions, gel diffusion precipitin reactions, immunodiffusion assays, fluorescent immunoassays, chemiluminescent immunoassays, phosphorescent immunoassays, anodic stripping voltammetric immunoassay and the like. Inventive in vitro diagnostic devices may also include any know devices currently available that utilize ion-selective electrode potentiometry, microfluids technology, fluorescence or chemiluminescence, or reflection technology that optically interprets color changes on a protein test strip. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation). It should be appreciated that at present none of the existing technologies present a method of detecting or measuring any of the ailments disclosed herein, nor does there exist any methods of using such in vitro diagnostic devices to detect any of the disclosed biomarkers to detect their associated injuries.

As used herein the term “diagnosing” means recognizing the presence or absence of a neurological or other condition such as an injury or disease. Diagnosing is optionally referred to as the result of an assay wherein a particular ratio or level of a biomarker is detected or is absent.

As used herein a “ratio” is either a positive ratio wherein the level of the target is greater than the target in a second sample or relative to a known or recognized baseline level of the same target. A negative ratio describes the level of the target as lower than the target in a second sample or relative to a known or recognized baseline level of the same target. A neutral ratio describes no observed change in target biomarker.

As used herein an injury is an alteration in cellular or molecular integrity, activity, level, robustness, state, or other alteration that is traceable to an event. Injury illustratively includes a physical, mechanical, chemical, biological, functional, infectious, or other modulator of cellular or molecular characteristics. An event is illustratively, a physical trauma such as an impact (percussive) or a biological abnormality such as a stroke resulting from either blockade or leakage of a blood vessel. An event is optionally an infection by an infectious agent. A person of skill in the art recognizes numerous equivalent events that are encompassed by the terms injury or event.

An injury is optionally a physical event such as a percussive impact. An impact is the like of a percussive injury such as resulting to a blow to the head that either leaves the cranial structure intact or results in breach thereof. Experimentally, several impact methods are used illustratively including controlled cortical impact (CCI) at a 1.6 mm depression depth, equivalent to severe TBI in human. This method is described in detail by Cox, C D, et al., J Neurotrauma, 2008; 25(11):1355-65. It is appreciated that other experimental methods producing impact trauma are similarly operable.

As used herein Coma shall mean the initial stage after a severe brain injury is a coma, a state of unconsciousness. People in a coma are unaware and unresponsive, but not asleep as there is no sleep-wake cycle. While in a coma, people are unable to speak, follow commands or open their eyes. As a person's GCS score improves, he or she is considered to be emerging from the coma. These changes usually take place gradually. For instance, eyes may open or there may be evidence of sleep cycles, but still no ability to speak or follow commands. As these abilities appear, most rehabilitation centers will use the Rancho Los Amigos Cognitive Scale (see separate document) to describe progress after this point.

As used herein Vegetative State shall mean the period where emergence from coma can seem to stop before the person becomes conscious. People in a vegetative state may open their eyes and have sleep-wake cycles, but are still unconscious. Although not considered to be in a coma, the patients remain totally unaware. In a vegetative state, any apparent signs of responding to surroundings are reflexes and not indications of awareness. The term permanent vegetative state is used only when a person is determined to be in a vegetative state for twelve months after trauma or three months after a brain injury that caused oxygen insufficiency. Always discuss with the physician questions about responses and awareness.

As used herein Minimally Conscious State refers to people who demonstrate some, but very little, awareness and responsiveness to their surroundings. Responses are typically inconsistent and thus not considered comatose or vegetative. As the name suggests, a person is considered conscious in this state. Occasionally, physicians may prescribe medicines that help stimulate the brain, especially if a person is not becoming more responsive with time. Some people do not progress beyond this stage in their recovery process. TBI may also result from stroke. Ischemic stroke is optionally modeled by middle cerebral artery occlusion (MCAO) in rodents. UCH-L1 protein levels, for example, are increased following mild MCAO which is further increased following severe MCAO challenge. Mild MCAO challenge may result in an increase of protein levels within two hours that is transient and returns to control levels within 24 hours. In contrast, severe MCAO challenge results in an increase in protein levels within two hours following injury and may be much more persistent demonstrating statistically significant levels out to 72 hours or more.

An exemplary process for detecting the presence or absence of a biomarker, alone or in combination, in a biological sample involves obtaining a biological sample from a subject, such as a human, contacting the biological sample with a compound or an agent capable of detecting of the marker being analyzed, illustratively including an antibody or aptamer, and analyzing binding of the compound or agent to the sample after washing. Those samples having specifically bound compound or agent express the marker being analyzed.

For example, in vitro techniques for detection of a marker illustratively include enzyme linked immunosorbent assays (ELISAs), radioimmuno assay, radioassay, western blot, Southern blot, northern blot, immunoprecipitation, immunofluorescence, mass spectrometry, RT-PCR, PCR, liquid chromatography, high performance liquid chromatography, enzyme activity assay, cellular assay, positron emission tomography, mass spectroscopy, combinations thereof, or other technique known in the art. Furthermore, in vivo techniques for detection of a marker include introducing a labeled agent that specifically binds the marker into a biological sample or test subject. For example, the agent can be labeled with a radioactive marker whose presence and location in a biological sample or test subject can be detected by standard imaging techniques. Optionally, the first biomarker specifically binding agent and the agent specifically binding at least one additional neuroactive biomarker are both bound to a substrate. It is appreciated that a bound agent assay is readily formed with the agents bound with spatial overlap, with detection occurring through discernibly different detection for first biomarker and each of at least one additional neuroactive biomarkers. A color intensity based quantification of each of the spatially overlapping bound biomarkers is representative of such techniques.

Any suitable molecule that can specifically bind to a biomarker and any suitable molecule that specifically binds one or more other biomarkers of a particular condition are operative in the invention to achieve a synergistic assay. A preferred agent for detecting the one or more biomarkers of a condition is an antibody capable of binding to the biomarker being analyzed. Preferably, an antibody is conjugated with a detectable label. Such antibodies can be polyclonal or monoclonal. An intact antibody, a fragment thereof (e.g., Fab or F(ab′)₂), or an engineered variant thereof (e.g., sFv) can also be used. Such antibodies can be of any immunoglobulin class including IgG, IgM, IgE, IgA, IgD and any subclass thereof. Antibodies for numerous inventive biomarkers are available from vendors known to one of skill in the art. Illustratively, antibodies directed to inventive biomarkers are available from Santa Cruz Biotechnology (Santa Cruz, Calif.). Exemplary antibodies operative herein are used to detect a biomarker of the disclosed conditions. In addition antigens to detect autoantibodies may also be used to detect chronic injury of the stated injuries and disorders.

An antibody is optionally labeled. A person of ordinary skill in the art recognizes numerous labels operable herein. Labels and labeling kits are commercially available optionally from Invitrogen Corp, Carlsbad, Calif. Labels illustratively include, fluorescent labels, biotin, peroxidase, radionucleotides, or other label known in the art. Alternatively, a detection species of another antibody or other compound known to the art is used as form detection of a biomarker bound by an antibody.

Antibody-based assays are preferred for analyzing a biological sample for the presence of a biomarker and one or more other biomarkers of a particular injury or condition. Suitable western blotting methods are described below in the examples section. For more rapid analysis (as may be important in emergency medical situations), immunosorbent assays (e.g., ELISA and RIA) and immunoprecipitation assays may be used. As one example, the biological sample or a portion thereof is immobilized on a substrate, such as a membrane made of nitrocellulose or PVDF; or a rigid substrate made of polystyrene or other plastic polymer such as a microtiter plate, and the substrate is contacted with an antibody that specifically binds GFAP, or one of the other neuroactive biomarkers under conditions that allow binding of antibody to the biomarker being analyzed. After washing, the presence of the antibody on the substrate indicates that the sample contained the marker being assessed. If the antibody is directly conjugated with a detectable label, such as an enzyme, fluorophore, or radioisotope, the presence of the label is optionally detected by examining the substrate for the detectable label. Alternatively, a detectably labeled secondary antibody that binds the marker-specific antibody is added to the substrate. The presence of detectable label on the substrate after washing indicates that the sample contained the marker.

Numerous permutations of these basic immunoassays are also operative in the invention. These include the biomarker-specific antibody, as opposed to the sample being immobilized on a substrate, and the substrate is contacted with a biomarker conjugated with a detectable label under conditions that cause binding of antibody to the labeled marker. The substrate is then contacted with a sample under conditions that allow binding of the marker being analyzed to the antibody. A reduction in the amount of detectable label on the substrate after washing indicates that the sample contained the marker.

Although antibodies are preferred for use in the invention because of their extensive characterization, any other suitable agent (e.g., a peptide, an aptamer, or a small organic molecule) that specifically binds a biomarker is optionally used in place of the antibody in the above described immunoassays. For example, an aptamer that specifically binds αII spectrin and/or one or more of its SBDPs might be used. Aptamers are nucleic acid-based molecules that bind specific ligands. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

A myriad of detectable labels that are operative in a diagnostic assay for biomarker expression are known in the art. Agents used in methods for detecting a biomarker are conjugated to a detectable label, e.g., an enzyme such as horseradish peroxidase. Agents labeled with horseradish peroxidase can be detected by adding an appropriate substrate that produces a color change in the presence of horseradish peroxidase. Several other detectable labels that may be used are known. Common examples of these include alkaline phosphatase, horseradish peroxidase, fluorescent compounds, luminescent compounds, colloidal gold, magnetic particles, biotin, radioisotopes, and other enzymes. It is appreciated that a primary/secondary antibody system is optionally used to detect one or more biomarkers. A primary antibody that specifically recognizes one or more biomarkers is exposed to a biological sample that may contain the biomarker of interest. A secondary antibody with an appropriate label that recognizes the species or isotype of the primary antibody is then contacted with the sample such that specific detection of the one or more biomarkers in the sample is achieved.

The present invention provides a step of comparing the quantity of one or more biomarkers to normal levels to determine the neurological condition of the subject. It is appreciated that selection of additional biomarkers allows one to identify the types of cells implicated in an abnormal organ or physical condition as well as the nature of cell death in the case of an axonal injury marker, namely an SBDP. The practice of an inventive process provides a test which can help a physician determine suitable therapeutics to administer for optimal benefit of the subject. While the neural data provided in the examples herein are provided with respect to a full spectrum of traumatic brain injury, neurotoxicity, and neuronal cell death, it is appreciated that these results are applicable to other ischemic events, neurodegenerative disorders, prion related disease, epilepsy, chemical etiology and peripheral nervous system pathologies. As is shown in the subsequently provided example data, a gender difference is unexpectedly noted in abnormal subject neurological condition.

The results of such a test using an in vitro diagnostic device can help a physician determine whether the administration a particular therapeutic or treatment regimen may be effective, and provide a rapid clinical intervention to the injury or disorder to enhance a patient's recovery.

It is appreciated that other reagents such as assay grade water, buffering agents, membranes, assay plates, secondary antibodies, salts, and other ancillary reagents are available from vendors known to those of skill in the art. Illustratively, assay plates are available from Corning, Inc. (Corning, N.Y.) and reagents are available from Sigma-Aldrich Co. (St. Louis, Mo.).

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

Various aspects of the present invention are illustrated by the following non-limiting examples. The examples are for illustrative purposes and are not a limitation on any practice of the present invention. It will be understood that variations and modifications can be made without departing from the spirit and scope of the invention. While the examples are generally directed to mammalian tissue, specifically, analyses of mouse tissue, a person having ordinary skill in the art recognizes that similar techniques and other techniques known in the art readily translate the examples to other mammals such as humans. Reagents illustrated herein are commonly cross reactive between mammalian species or alternative reagents with similar properties are commercially available, and a person of ordinary skill in the art readily understands where such reagents may be obtained. Variations within the concepts of the invention are apparent to those skilled in the art.

### Example 1

### Materials for Biomarker Analyses

Illustrative reagents used in performing the subject invention include Sodium bicarbonate (Sigma Cat #: C-3041), blocking buffer (Startingblock T20-TBS) (Pierce Cat#: 37543), Tris buffered saline with Tween 20 (TBST; Sigma Cat #: T-9039). Phosphate buffered saline (PBS; Sigma Cat #: P-3813); Tween 20 (Sigma Cat #: P5927); Ultra TMB ELISA (Pierce Cat #: 34028); and Nunc maxisorp ELISA plates (Fisher). Monoclonal and polyclonal GFAP and UCH-L1 antibodies are made in-house or are obtained from Santa Cruz Biotechnology, Santa Cruz, Calif. Antibodies directed to α-II spectrin and breakdown products as well as to MAP2 are available from Santa Cruz Biotechnology, Santa Cruz, Calif. Labels for antibodies of numerous subtypes are available from Invitrogen, Corp., Carlsbad, Calif. Protein concentrations in biological samples are determined using bicinchoninic acid microprotein assays (Pierce Inc., Rockford, Ill., USA) with albumin standards. All other necessary reagents and materials are known to those of skill in the art and are readily ascertainable.

### Example 2

### Biomarker Assay Development

Anti-biomarker specific rabbit polyclonal antibody and monoclonal antibodies are produced in the laboratory. To determine reactivity specificity of the antibodies to detect a target biomarker a known quantity of isolated or partially isolated biomarker is analyzed or a tissue panel is probed by western blot. An indirect ELISA is used with the recombinant biomarker protein attached to the ELISA plate to determine optimal concentration of the antibodies used in the assay. Microplate wells are coated with rabbit polyclonal anti-human biomarker antibody. After determining the concentration of rabbit anti-human biomarker antibody for a maximum signal, the lower detection limit of the indirect ELISA for each antibody is determined. An appropriate diluted sample is incubated with a rabbit polyclonal antihuman biomarker antibody for 2 hours and then washed. Biotin labeled monoclonal anti-human biomarker antibody is then added and incubated with captured biomarker. After thorough wash, streptavidin horseradish peroxidase conjugate is added. After 1 hour incubation and the last washing step, the remaining conjugate is allowed to react with substrate of hydrogen peroxide tetramethyl benzadine. The reaction is stopped by addition of the acidic solution and absorbance of the resulting yellow reaction product is measured at 450 nanometers. The absorbance is proportional to the concentration of the biomarker. A standard curve is constructed by plotting absorbance values as a function of biomarker concentration using calibrator samples and concentrations of unknown samples are determined using the standard curve.

### Example 3

### TBI Patient Samples

Subjects with suspected TBI are enrolled at several investigational sites globally. All Subjects receive standard of care treatment when presenting to the investigational site. Biological samples of blood, urine, saliva and CSF are collected from the subjects at specified timepoints. Inclusion criteria for the Subjects include 1) The Subject is at least 18 years of age at screening (has had their 18th birthday) and no more than 80 years of age (did not have their 81st birthday); 2) the Subject received an accelerated or decelerated closed injury to the head (this includes head injuries inflicted by blunt force mechanism) self-reported or witnessed; 3) the biological samples of blood urine and saliva are able to be collected within four (4) hours after injury; 4) the Subject is admitted with an initial Glasgow Coma Scale score of 3-8 (severe TBI), or from 5-15 (mild or moderate TBI); 5) the Subject is willing to undergo a computerized tomography (CT) of the head; 6) proper informed consent from patient or guardian. Severe TBI patients may be admitted if in a coma or a vegetative state, while mild to moderate patients may be admitted in a minimally conscious state or suffering from post-traumatic amnesia, retrograde or otherwise. Notwithstanding, the Glasgow coma score upon admission to a investigational site shall control which severity of injury the Subject is included. Follow up visits at 7 and 35 days after injury are included in the sample cohort, where again biological samples are drawn. Upon enrollment into the study, further neurocognitive tests such as RBANS, King Devic, GOAT, BESS and other tests measuring the neurocognitive abilities of a Subject are employed. These tests are also administered during patient follow-up visits to track a patients recovery and correlate with chronic biomarker measurement.

### Example 4

### Stroke Patient Samples

Subjects with suspected stroke are enrolled at several investigational sites globally. All Subjects receive standard of care treatment when presenting to the investigational site. Biological samples of blood, urine, saliva and CSF are collected from the subjects at specified time points. Inclusion criteria for the Subjects include 1) The Subject is at least 18 years of age at screening (has had their 18th birthday) and no more than 80 years of age (did not have their 81st birthday); 2) the Subjects primary diagnosis is ischemic or hemorrhagic stroke, self-reported or witnessed; 3) the biological samples of blood urine and saliva are able to be collected within four (4) hours after injury; 4) the Subject is willing to undergo a computerized tomography (CT) of the head; 5) proper informed consent from patient or guardian.

### Example 5

### Normal Patient Samples

Normal Subjects without any known or suspected TBI, stroke or other conditions which may alter protein biomarker levels are enrolled at several investigational sites globally. All Subjects receive standard screening to ensure that no medications or ailments are experienced by the patients prior to enrollment into the study. Biological samples of blood, urine, saliva and CSF are collected from the subjects upon enrollment. Inclusion criteria for the Subjects include 1) The Subject is at least 18 years of age at screening (has had their 18th birthday) and no more than 80 years of age (did not have their 81st birthday); 2) the Subject is screened and found to not be taking medications or suffering from any neurological injury, neurological disorder or neurotoxicity; 3) proper informed consent from patient or guardian

### Example 6

### Analysis of Mild, Moderate and Severe TBI Markers

Accumulation of novel neural markers glial fibrillary acidic protein (GFAP) or one of its breakdown products, Ubiquitin carboxyl-terminal hydrolase L1 (UCH-L1), neuron specific enolase (NSE), Microtubule-associated protein 2 (MAP2), myelin basic protein (MBP), α-II spectrin breakdown product (SBDP) preferably SBDP150, SBDP150i SBDP145, SBDP120, S100 calcium binding protein B (S100b), vesicular membrane protein neurensin-1 (p24), collapsin response mediated proteins (CRMP's) and breakdown products thereof, or synaptotagmin and breakdown products thereof are analyzed in the biological samples taken after TBI using the inventive in vitro diagnostic devices. Normal patient samples are also analyzed for the same biomarkers, and a normal metric is calculated to indicate a non-injury state. The metric is then incorporated into the in vitro diagnostic device either through a computer algorithm, or in the event of a calorimetric indication, the dyes are activated indicating injury when the level of the measured biomarker is higher than what is determined in the normal metric.

Prior to analysis, an assay is developed using a detection and capture antibody, each antibody being specific to the biomarker intended to be measured. For example, for GFAP a monoclonal/monoclonal pair (capture/detection) is used to detect the level of biomarkers. Notwithstanding, similar results are achieved through the use of a monoclonal/polyclonal pair, a polyclonal monoclonal pair, and a polyclonal/polyclonal pair. The assay is optimized and tested using a calibrator and spiked serum to ensure that assay can measure known positive and known negative controls and detect the levels of known proteins within 1 picogram/mL detection sensitivity. The assay is incorporated into an in vitro diagnostic device using a cartridge or other disposable, whereby the cartridge contains the assay and a biological sample collection chamber for receiving the biological sample. The present invention further incorporates by reference the antibody and detection methods for the claimed biomarkers being used in the device for the specific indication disclosed therein presented in US 2007/0003982 A1, US 2005/0260697 A1, US 2009/0317805 A1, US 2005/0260654 A1, US 2009/0087868 A1, US 2010/0317041 A1, US 2011/0177974 A1, US 2010/0047817 A1, US 2012/0196307 A1, US 2011/0082203 A1, US 2011/0097392 A1, US 2011/0143375 A1, US 2013/0029859 A1, US 2012/0202231 A1, and US 2013/0022982 A1 and application Ser. No. 13/470,079. The in vitro diagnostic devices used in this example have incorporated assays contained therein, which assays may be substituted herein using the methods therein contained.

### Example 7

### Analysis of Neurotoxic Marker

Accumulation of novel markers indicating neurotoxic insult such as ubiquitin carboxyl-terminal hydrolase-L1 (UCH-L1); spectrin; a spectrin breakdown product (SBDP); MAP1, MAP2; GFAP, ubiquitin carboxyl-terminal esterase; ubiquitin carboxyl-terminal hydrolase; a neuronally-localized intracellular protein; MAP-tau; C-tau; Poly (ADP-ribose) polymerase (PARP); a collapsin response mediator protein, synaptotagmin, βIII-tubulin, S100β; neuron-specific enolase, neurofilament protein light chain, nestin, α-internexin; breakdown products thereof, post-translationally modified forms thereof, derivatives thereof, and combinations thereof, are analyzed in the biological samples taken after TBI using the inventive in vitro diagnostic devices. Normal patient samples are also analyzed for the same biomarkers, and a normal metric is calculated to indicate a non-injury state. The metric is then incorporated into the in vitro diagnostic device either through a computer algorithm, or in the event of a calorimetric indication, the dyes are activated indicating injury when the level of the measured biomarker is higher than what is determined in the normal metric.

Prior to analysis, an assay is developed using a detection and capture antibody, each antibody being specific to the biomarker intended to be measured. For example, for SBDP-145 a monoclonal/monoclonal pair (capture/detection) is used to detect the level of biomarkers. Notwithstanding, similar results are achieved through the use of a monoclonal/polyclonal pair, a polyclonal monoclonal pair, and a polyclonal/polyclonal pair. The assay is optimized and tested using a calibrator and spiked serum to ensure that assay can measure known positive and known negative controls and detect the levels of known proteins within 1 picogram/mL detection sensitivity. The assay is incorporated into an in vitro diagnostic device using a cartridge or other disposable, whereby the cartridge contains the assay and a biological sample collection chamber for receiving the biological sample. The present invention further incorporates by reference the antibody and detection methods for the claimed biomarkers being used in the device for the specific indication disclosed therein presented in US 2013/0029362 A1. The in vitro diagnostic devices used in this example have incorporated assays contained therein, which assays may be substituted herein using the methods therein contained.

### Example 8

### Severe Traumatic Brain Injury Study

A study is conducted that included 46 human subjects suffering severe traumatic brain injury. Each of these subjects is characterized by being over age 18, having a GCS of less than or equal to 8 and required ventriculostomy and neuromonitoring as part of routine care. A control group A, synonymously detailed as CSF controls, included 10 individuals also being over the age of 18 or older and no injuries. Samples are obtained during spinal anesthesia for routine surgical procedures or access to CSF associated with treatment of hydrocephalus or meningitis. A control group B, synonymously described as normal controls, totaled 64 individuals, each age 18 or older and experiencing multiple injuries without brain injury. Further details with respect to the demographics of the study are provided in Table 1.

TABLE 1
Subject Demographics for Severe Traumatic Brain Injury Study

| | | TBI | CSF Controls | Normal Controls |
|---|---|---|---|---|
| Number | | 46 | 10 | 64 |
| | Males | 34 (73.9%) | 29 (65.9%) | 26 (40.6%) |
| | Females | 12 (26.1%) | 15 (34.1%) | 38 (59.4% |
| Age: | Average | 50.2 | 58.2 1, 2 | 30.09 2, 3 |
| | Std Dev | 19.54 | 20.52 | 15.42 |
| | Minimum | 19 | 23 | 18 |
| | Maximum | 88 | 82 | 74 |
| Race: | Caucasian | | | |
| | Black | 45 | 38 (86.4%) | 52 (81.2%) |
| | Asian | 1 | 6 (13.6) | 4 (6.3%) |
| | Other | | | 7 (10.9%) |
| | | | | 1 (1.6%) |

GCS in Emergency Department

| | |
|---|---|
| Average | 5.3 |
| Std Dev | 1.9 |

The level of biomarkers found in the first available CSF and serum samples obtained in the study are provided in the Figures. The average first CSF sample collected as detailed in the Figures is 11.2 hours while the average time for collection of a serum sample subsequent to injury event as per the Figures is 10.1 hours. The quantity of each of the biomarkers of UCH-L1, MAP2, SBDP145, SBDP120, and GFAP are provided for each sample for the cohort of traumatic brain injury sufferers as compared to a control group. The diagnostic utility of the various biomarkers within the first 12 hours subsequent to injury based on a compilation of CSF and serum data is provided in the Figures and indicates in particular the value of GFAP as well as that of additional markers UCH-L1 and the spectrin breakdown products. Elevated levels of UCH-L1 are indicative of the compromise of neuronal cell body damage while an increase in SPDP145 with a corresponding decrease in SBDP120 is suggestive of acute axonal necrosis.

One subject from the traumatic brain injury cohort is a 52 year old Caucasian woman who had been involved in a motorcycle accident while not wearing a helmet. Upon admission to an emergency room her GCS is 3 and during the first 24 hours subsequent to trauma her best GCS is 8. After 10 days her GCS is 11. CT scanning revealed SAH and facial fractures with a Marshall score of 11 and a Rotterdam score of 2. Ventriculostomy is removed after 5 years and an overall good outcome is obtained. Arterial blood pressure (MABP), intracranial pressure (ICP) and cerebral profusion pressure (CPP) for this sufferer of traumatic brain injury as a function of time is depicted in the Figures. A possible secondary insult is noted at approximately 40 hours subsequent to the injury as noted by a drop in MABP and CPP. The changes in concentration of inventive biomarkers per CSF and serum samples from this individual are noted in the Figures. These results include a sharp increase in GFAP in both the CSF and serum as well as the changes in the other biomarkers depicted in the Figures and provide important clinical information as to the nature of the injury and the types of cells involved, as well as modes of cell death associated with the spectrin breakdown products.

Another individual of the severe traumatic brain injury cohort included a 51 year old Caucasian woman who suffered a crush injury associated with a horse falling on the individual. GCS on admission to emergency room is 3 with imaging analysis initially being unremarkable with minor cortical and subcortical contusions. MRI on day 5 revealed significant contusions in posterior fossa. The Marshall scale at that point is indicated to be 11 with a Rotterdam scale score of 3. The subject deteriorated and care is withdrawn 10 days after injury. The CSF and serum values for this individual during a period of time are provided in the Figures.

Based on the sandwich ELISA testing, GFAP values as a function of time are noted to be markedly elevated relative to normal controls (control group B) as a function of time.

The concentration of spectrin breakdown products, MAP2 and UCH-L1 as a function of time subsequent to traumatic brain injury has been reported elsewhere as exemplified in U.S. Pat. Nos. 7,291,710 and 7,396,654 each of which is incorporated herein by reference.

An analysis is performed to evaluate the ability of biomarkers measured in serum to predict TBI outcome, specifically GCS. Stepwise regression analysis is the statistical method used to evaluate each of the biomarkers as an independent predictive factor, along with the demographic factors of age and gender, and also interactions between pairs of factors. Interactions determine important predictive potential between related factors, such as when the relationship between a biomarker and outcome may be different for men and women, such a relationship would be defined as a gender by biomarker interaction.

The resulting analysis identified biomarkers UCH-L1, MAP2, and GFAP as being statistically significant predictors of GCS (Table 2, 3). Furthermore, GFAP is shown to have improved predictability when evaluated in interaction with UCH-L1 and gender (Table 4, 5).

TABLE 2
Stepwise Regression Analysis 1 - Cohort includes:
All Subjects >=18 Years Old
Summary of Stepwise Selection - 48 Subjects

| Variable | Parameter | Model | | |
|---|---|---|---|---|
| Step Entered | Estimate | R-Square | F Value | p-value |
| Intercept | 13.02579 | | | |
| 2 SEXCD | −2.99242 | 0.1580 | 7.29 | 0.0098 |
| 1 CSF_UCH_L1 | −0.01164 | 0.2519 | 11.54 | 0.0015 |
| 3 Serum_MAP_2 | 0.96055 | 0.3226 | 4.59 | 0.0377 |

TABLE 3
Stepwise Regression Analysis 2 - Cohort includes:
TBI Subjects >=18 Years Old
Summary of Stepwise Selection - 39 Subjects

| Variable | Parameter | Model | | |
|---|---|---|---|---|
| Step Entered | Estimate | R-Square | F Value | p-value |
| Intercept | 5.73685 | | | |
| 1 Serum_UCH_L1 | −0.30025 | 0.0821 | 8.82 | 0.0053 |
| 2 Serum_GFAP | 0.12083 | 0.1973 | 5.16 | 0.0291 |

TABLE 4
Stepwise Regression Analysis 1 - Cohort includes:
TBI and A Subjects >=18 Years Old
Summary of Stepwise Selection - 57 Subjects

| Variable | Parameter | Model | | |
|---|---|---|---|---|
| Step Entered | Estimate | R-Square | F Value | p-value |
| Intercept | 8.04382 | | | |
| 1 Serum_UCH_L | −0.92556 | 0.1126 | 12.90 | 0.0007 |
| 2 Serum_MAP_2 | 1.07573 | 0.2061 | 5.79 | 0.0197 |
| 3 Serum_UCH-L1 + | 0.01643 | 0.2663 | 4.35 | 0.0419 |
| Serum_GFAP | | | | |

TABLE 5
Stepwise Regression Analysis 2 - Cohort includes:
TBI Subjects >=18 Years Old
Summary of Stepwise Selection - 44 Subjects

| Variable | Parameter | Model | | |
|---|---|---|---|---|
| Step Entered | Estimate | R-Square | F Value | p-value |
| Intercept | 5.50479 | | | |
| 1 Serum_UCH_L1 | −0.36311 | 0.0737 | 11.95 | 0.0013 |
| 2 SEX_Serum_GFAP | 0.05922 | 0.1840 | 5.09 | 0.0296 |
| 3 Serum_MAP_2 | 0.63072 | 0.2336 | 2.59 | 0.1157 |

### Example 9

The study of Example 8 is repeated with a moderate traumatic brain injury cohort characterized by GCS scores of between 9 and 11, as well as a mild traumatic brain injury cohort characterized by GCS scores of 12-15. Blood samples are obtained from each patient on arrival to the emergency department of a hospital within 2 hours of injury and measured by ELISA for levels of GFAP in nanograms per milliliter. The results are compared to those of a control group who had not experienced any form of injury. Secondary outcomes included the presence of intracranial lesions in head CT scans.

Over 3 months 53 patients are enrolled: 35 with GCS 13-15, 4 with GCS 9-12 and 14 controls. The mean age is 37 years (range 18-69) and 66% are male. The mean GFAP serum level is 0 in control patients, 0.107 (0.012) in patients with GCS 13-15 and 0.366 (0.126) in GCS 9-12 (P<0.001). The difference between GCS 13-15 and controls is significant at P<0.001. In patients with intracranial lesions on CT GFAP levels are 0.234 (0.055) compared to 0.085 (0.003) in patients without lesions (P<0.001). There is a significant increase in GFAP in serum following a MTBI compared to uninjured controls in both the mild and moderate groups. GFAP is also significantly associated with the presence of intracranial lesions on CT.

The Figures shows GFAP concentration for controls as well as individuals in the mild/moderate traumatic brain injury cohort as a function of CT scan results upon admission and 24 hours thereafter. Simultaneous assays are performed in the course of this study for UCH-L1 biomarker. The UCH-L1 concentration derived from the same samples as those used to determine GFAP is provided the Figures. The concentration of UCH-L1 and GFAP as well as a biomarker not selected for diagnosis of neurological condition, S100b, is provided as a function of injury magnitude between control, mild, and moderate traumatic brain injury as shown in The Figures. The simultaneous analyses of UCH-L1 and GFAP from these patients illustrates the synergistic effect of the inventive process in allowing an investigator to simultaneously diagnose traumatic brain injury as well as discern the level of traumatic brain injury between mild and moderate levels of severity. The Figures show the concentration of the same markers as depicted in the Figures with respect to initial evidence upon hospital admission as to lesions in tomography scans illustrating the high confidence in predictive outcome of the inventive process. The Figures show that both NSE and MAP2 are elevated in subjects with MTBI in serum both at admission and at 24 hours of follow up. These data demonstrate a synergistic diagnostic effect of measuring multiple biomarkers such as GFAP, UCH-L1, NSE, and MAP2 in a subject.

Through the simultaneous measurement of multiple biomarkers such as UCH-L1, GFAP, NSE, and MAP2, rapid and quantifiable determination as to the severity of the brain injury is obtained consistent with GSC scoring and CT scanning yet in a surprisingly more quantifiable, expeditious and economic process. Additionally, with a coupled assay for biomarkers indicative of neurological condition, the nature of the neurological abnormality is assessed and in this particular study suggestive of neuronal cell body damage. As with severe traumatic brain injury, gender variations are noted suggesting a role for hormonal anti-inflammatories as therapeutic candidates. A receiver operating characteristic (ROC) modeling of UCH-L1, GFAP and SBDP145 post TBI further supports the value of simultaneous measurement of these biomarkers, as shown in the Figures.

In addition, The Figures show that several brain biomarkers (GFAP, UCH-L1 and MAP2) in stroke patients' plasma. Samples are collected with an average post-injury time 24.2 hr (range 18-30 h). Top panel shows GFAP, UCH-L1 and MAP2 levels in stroke (n=11) versus normal controls (n=30). Bottom panel further shows that UCH-L1 is elevated with both hemorrhagic and ischemic stroke populations when compared to normal control plasma.

### Other Embodiments

Patent documents and publications mentioned in the specification are indicative of the levels of those skilled in the art to which the invention pertains. These documents and publications are incorporated herein by reference to the same extent as if each individual document or publication is specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. An in vitro diagnostic device for detecting a neural injury or neuronal disorder in a subject, the device comprising:
a sample chamber for holding a first biological sample collected from the subject;
an assay module in fluid communication with said sample chamber, said assay module containing an agent for detecting one or more biomarkers of a neural injury or neuronal disorder selected from the group consisting of (GFAP) or one of its breakdown products, Ubiquitin carboxyl-terminal hydrolase L1 (UCH-L1), neuron specific enolase (NSE), Microtubule-associated protein 2 (MAP2), myelin basic protein (MBP), α-II spectrin breakdown product (SBDP) preferably SBDP150, SBDP150i SBDP145, SBDP120, S100 calcium binding protein B (S100b), vesicular membrane protein neurensin-1 (p24), collapsin response mediated proteins (CRMP's) and breakdown products thereof, or synaptotagmin and breakdown products thereof, wherein said assay module analyzes the first biological sample to detect the amount of the one or more biomarker present in said sample;
a user interface, wherein said user interface relates the amount of the one or more biomarker measured in the assay module to detecting a neural injury or neuronal disorder in the subject or the severity of neural injury or neuronal disorder in the subject.

2. The device of claim 1, wherein the neural injury or neuronal disorder is one of: stroke, epilepsy, hypoxic ischemic encephalopathy (HIE), chronic traumatic encephalopathy (CTE), Alzheimer's disease (AD), Parkinson's disease (PD), traumatic brain injury (TBI), neurotoxicity, spinal cord injury (SCI) or neural cell damage.

3. The device of claim 1 wherein said assay module further comprises at least one additional agent selective to measure for at least one additional biomarker selected from the group consisting of: (GFAP) or one of its breakdown products, Ubiquitin carboxyl-terminal hydrolase L1 (UCH-L1), neuron specific enolase (NSE), Microtubule-associated protein 2 (MAP2), myelin basic protein (MBP), α-II spectrin breakdown product (SBDP) preferably SBDP150, SBDP150i SBDP145, SBDP120, S100 calcium binding protein B (S100b), vesicular membrane protein neurensin-1 (p24), collapsin response mediated proteins (CRMP's) and breakdown products thereof, or synaptotagmin and breakdown products thereof.

4. The device of claim 1 wherein the first biological sample is selected from the group consisting of blood, blood plasma, serum, sweat, saliva, cerebrospinal fluid (CSF) and urine.

5. The device of claim 1 wherein said assay further comprises a dye providing a colorimetric change in response to the one or more biomarker present in the first biological sample.

6. The device of claim 1 wherein said assay module is an immunoassay.

7. The device of claim 6 wherein the immunoassay is an ELISA.

8. The device of claim 1, wherein said agent is an antibody or a protein.

9. The device of claim 1, further comprising a power supply and a data processing module in operable communication with said power supply and said assay module wherein said data processing module has an output that relates to detecting the neural injury or neuronal disorder in the subject, the output displaying the amount of the one or more biomarker measured in said sample, the output displaying the presence or absence of a neural injury or neuronal disorder, or the output displaying the severity of neural injury or neuronal disorder.

10. The device of claim 9, further comprising analyzing a second biological sample obtained from the subject, at some time after the first sample is collected, wherein if the device detects a decreased amount of the one or more biomarker in the second sample relative to the first sample a recovery output is provided by the data processing module.

11. The device of claim 9 further comprising a display in electrical communication with said data processing module and displaying the output as at least one of an amount of the one or more biomarker, a comparison between the amount of the one or more biomarker and a control, presence of the neural injury or neuronal disorder, or severity of the neural injury or neuronal disorder.

12. The device of claim 9 further comprising a transmitter for communicating the output to a remote location.

13. The device of claim 9 wherein the output is digital.

14. A method for using an in vitro diagnostic device for detecting a neural injury or neuronal disorder in a subject, the method comprising:
calibrating an in vitro diagnostic device incorporating an assay for measuring one or more biomarkers of a neural injury or neuronal disorder in a biological sample, the one or more biomarkers selected from the group consisting of (GFAP) or one of its breakdown products, Ubiquitin carboxyl-terminal hydrolase L1 (UCH-L1), neuron specific enolase (NSE), Microtubule-associated protein 2 (MAP2), myelin basic protein (MBP), α-II spectrin breakdown product (SBDP) preferably SBDP150, SBDP150i SBDP145, SBDP120, S100 calcium binding protein B (S100b), vesicular membrane protein neurensin-1 (p24), collapsin response mediated proteins (CRMP's) and breakdown products thereof, or synaptotagmin and breakdown products thereof;
obtaining a biological sample from a subject;
applying said sample to said in vitro diagnostic device wherein said assay includes reagents to determine the amount of the one or more biomarker present in said sample, wherein said device provides an output which relates the amount of the one or more biomarker detected to a neural injury or neuronal disorder, or lack thereof, in the subject.

15. The method of claim 14 further comprising:
calibrating an in vitro diagnostic device incorporating an assay for additionally measuring at least one additional biomarker selected from the group consisting of: (GFAP) or one of its breakdown products, Ubiquitin carboxyl-terminal hydrolase L1 (UCH-L1), neuron specific enolase (NSE), Microtubule-associated protein 2 (MAP2), myelin basic protein (MBP), α-II spectrin breakdown product (SBDP) preferably SBDP150, SBDP150i SBDP145, SBDP120, S100 calcium binding protein B (S100b), vesicular membrane protein neurensin-1 (p24), collapsin response mediated proteins (CRMP's) and breakdown products thereof, or synaptotagmin and breakdown products thereof;
applying said sample to said in vitro diagnostic device wherein said assay includes reagents to determine the amount of the additional biomarker present in said sample, wherein said device provides an output which relates the amount of the additional biomarker detected, alone or in synergistic combination with the one or more biomarker, to a neural injury or neuronal disorder, or lack thereof, in the subject

16. A method of treating a neural injury or neuronal disorder in a subject:
calibrating an in vitro diagnostic device incorporating an assay for measuring for one or more biomarkers in a biological sample, the one or more biomarkers selected from the group consisting of (GFAP) or one of its breakdown products, Ubiquitin carboxyl-terminal hydrolase L1 (UCH-L1), neuron specific enolase (NSE), Microtubule-associated protein 2 (MAP2), myelin basic protein (MBP), α-II spectrin breakdown product (SBDP) preferably SBDP150, SBDP150i SBDP145, SBDP120, S100 calcium binding protein B (S100b), vesicular membrane protein neurensin-1 (p24), collapsin response mediated proteins (CRMP's) and breakdown products thereof, or synaptotagmin and breakdown products thereof;
obtaining a biological sample from a subject;
applying said sample to said in vitro diagnostic device wherein said assay includes reagents to determine the amount of the one or more biomarker present in said sample, wherein said device provides an output which relates the amount of the one or more biomarker detected to a neural injury or neuronal disorder, or lack thereof, in the subject, wherein if said output of said in vitro diagnostic device relates the amount of the one or more biomarker to a neuronal injury or neuronal disorder a therapeutic intervention is employed to treat injury and/or inhibit injury progression.

17. A process for electronically diagnosing a neurological condition in a subject, the process comprising:
an input signal from an assay module that has measured an amount of a biomarker of a neurological condition in a biological sample;
a software package providing instructions to a central processing unit for receiving and processing the input signal, comparing the input signal to a database of biomarker levels of a neurological condition to determine if the amount if the input is greater than or less than the database amount stored on a memory unit of a data processing module, and translating the input data into usable indication of the presence or absence of the neurological condition; and
communicating the usable indication to a graphical user interface to display the indication.

18. The process of claim 17 further comprising a network for communicating the usable indication to a remote display database, or computer terminal.

19. The process of claim 17 further comprising saving the usable indication in machine readable format to the memory unit of the data processing module.

20. The process of claim 17 wherein the comparison step is performed by a CPU receiving instructions from a software application.

21. The process of claim 17 wherein the database amount is a threshold level, predetermined for each biomarker respectively, is based on a known positive level of the biomarker, is a known negative level of the biomarker, or is the amount of the biomarker measured in normal control.

22. The process of claim 17 wherein the biomarker of the neurological condition is one or more biomarkers selected from the group consisting of: (GFAP) or one of its breakdown products, Ubiquitin carboxyl-terminal hydrolase L1 (UCH-L1), neuron specific enolase (NSE), Microtubule-associated protein 2 (MAP2), myelin basic protein (MBP), α-II spectrin breakdown product (SBDP) preferably SBDP150, SBDP150i SBDP145, SBDP120, S100 calcium binding protein B (S100b), vesicular membrane protein neurensin-1 (p24), collapsin response mediated proteins (CRMP's) and breakdown products thereof, or synaptotagmin and breakdown products thereof

23. The process of claim 17 wherein the usable indication is the measured amount of the biomarker present in the sample, an indication of the presence or absence of a neurological condition, the type of neurological condition, or the severity of a neurological condition.

24. The process of claim 17 wherein the input is two or more inputs received from at least one assay module of two or more biomarkers measured by the assay module.

25. The process of claim 1y, wherein the neural is one of: stroke, epilepsy, hypoxic ischemic encephalopathy (HIE), chronic traumatic encephalopathy (CTE), Alzheimer's disease (AD), Parkinson's disease (PD), traumatic brain injury (TBI), neurotoxicity, spinal cord injury (SCI) or neural cell damage.
